SEQUENCE LISTING (14 sequences)

SEQ ID NO: 1 - DNA, 4716 bp, homo sapiens
  CDS (82)..(4158): TN-R
ccttggtttccgttgcagattcccacaactccatgctgtgtgctgcaggctggtcctgaacccagatctctggctgagaggatgggggcagatggggaaacagtggttctgaagaacatgctcattggcgtcaacctgatccttctgggctccatgatcaagccttcagagtgtcagctggaggtcaccacagaaagggtccagagacagtcagtggaggaggagggaggcattgccaactacaacacgtccagcaaagagcagcctgtggtcttcaaccacgtgtacaacattaacgtgcccttggacaacctctgctcctcagggctagaggcctctgctgagcaggaggtgagtgcagaagacgagactctggcagagtacatgggccagacctcagaccacgagagccaggtcacctttacacacaggatcaacttccccaaaaaggcctgtccatgtgccagttcagcccaggtgctgcaggagctgctgagccggatcgagatgctggagagggaggtgtcggtgctgcgagaccagtgcaacgccaactgctgccaagaaagtgctgccacaggacaactggactatatccctcactgcagtggccacggcaactttagctttgagtcctgtggctgcatctgcaacgaaggctggtttggcaagaattgctcggagccctactgcccgctgggttgctccagccggggggtgtgtgtggatggccagtgcatctgtgacagcgaatacagcggggatgactgttccgaactccggtgcccaacagactgcagctcccgggggctctgcgtggacggggagtgtgtctgtgaagagccctacactggcgaggactgcagggaactgaggtgccctggggactgttcggggaaggggagatgtgccaacggtacctgtttatgcgaggagggctacgttggtgaggactgcggccagcggcagtgtctgaatgcctgcagtgggcgaggacaatgtgaggaggggctctgcgtctgtgaagagggctaccagggccctgactgctcagcagttgcccctccagaggacttgcgagtggctggtatcagcgacaggtccattgagctggaatgggacgggccgatggcagtgacggaatatgtgatctcttaccagccgacggccctggggggcctccagctccagcagcgggtgcctggagattggagtggtgtcaccatcacggagctggagccaggtctcacctacaacatcagcgtctacgctgtcattagcaacatcctcagccttcccatcactgccaaggtggccacccatctctccactcctcaagggctacaatttaagacgatcacagagaccaccgtggaggtgcagtgggagcccttctcattttccttcgatgggtgggaaatcagcttcattccaaagaacaatgaagggggagtgattgctcaggtccccagcgatgttacgtcctttaaccagacaggactaaagcctggggaggaatacattgtcaatgtggtggctctgaaagaacaggcccgcagcccccctacctcggccagcgtctccacagtcattgacggccccacgcagatcctggttcgcgatgtctcggacaccgtggcttttgtggagtggattccccctcgagccaaagtcgatttcattcttttgaaatatggcctggtgggcggggaaggtgggaggaccaccttccggctgcagcctcccctgagccaatactcagtgcaggccctgcggcctggctcccgatacgaggtgtcagtcagtgccgtccgagggaccaacgagagcgattctgccaccactcagttcacaacagagatcgatgcccccaagaacttgcgagttggttctcgcacagcaaccagccttgacctcgagtgggataacagtgaagccgaagttcaggagtacaaggttgtgtacagcaccctggcgggtgagcaatatcatgaggtactggtccccaggggcattggtccaaccaccagggccaccctgacagatctggtacctggcactgagtatggagttggaatatctgccgtcatgaactcacagcaaagcgtgccagccaccatgaatgccaggactgaacttgacagtccccgagacctcatggtgacagcctcctcggagacctccatctccctcatctggaccaaggccagtggccccattgaccactaccgaattacctttaccccatcctctgggattgcctcagaagtcaccgtacccaaggacaggacctcatacacactaacagatctagagcctggggcagagtacatcatttccgtcactgctgagaggggtcggcagcagagcttggagtccactgtggatgctttcacaggcttccgtcccatctctcatctgcacttttctcatgtgacctcctccagtgtgaacatcacttggagtgatccatctcccccagcagacagactcattcttaactacagccccagggatgaggaggaagagatgatggaggtctccctggatgccaccaagaggcatgctgtcctgatgggcctgcaaccagccacagagtatattgtgaaccttgtggctgtccatggcacagtgacctctgagcccattgtgggctccatcaccacaggaattgatcccccaaaagacatcacaattagcaatgtgaccaaggactcagtgatggtctcctggagccctcctgttgcatctttcgattactaccgagtatcatatcgacccacccaagtgggacgactagacagctcagtggtgcccaacactgtgacagaattcaccatcaccagactgaacccagctaccgaatacgaaatcagcctcaacagcgtgcggggcagggaggaaagcgagcgcatctgtactcttgtgcacacagccatggacaaccctgtggatctgattgctaccaatatcactccaacagaagccctgctgcagtggaaggcaccagtgggtgaggtggagaactacgtcattgttcttacacactttgcagtcgctggagagaccatccttgttgacggagtcagtgaggaatttcggcttgttgacctgcttcctagcacccactatactgccaccatgtatgccaccaatggacctctcaccagtggcaccatcagcaccaacttttctactctcctggaccctccggcaaacctgacagccagtgaagtcaccagacaaagtgccctgatctcctggcagcctcccagggcagagattgaaaattatgtcttgacctacaaatccaccgacggaagccgcaaggagctgattgtggatgcagaagacacctggattcgactggagggcctgttggagaacacagactacacggtgctcctgcaggcagcacaggacaccacgtggagcagcatcacctccaccgctttcaccacaggaggccgggtgttccctcatccccaagactgtgcccagcatttgatgaatggagacactttgagtggggtttaccccatcttcctcaatggggagctgagccagaaattacaagtgtactgtgatatgaccaccgacgggggcggctggattgtattccagaggcggcagaatggccaaactgattttttccggaaatgggctgattaccgtgttggcttcgggaacgtggaggatgagttctggctggggctggacaatatacacaggatcacatcccagggccgctatgagctgcgcgtggacatgcgggatggccaggaggccgccttcgcctcctacgacaggttctctgtcgaggacagcagaaacctgtacaaactccgcataggaagctacaacggcactgcgggggactccctcagctatcatcaaggacgccctttctccacagaggatagagacaatgatgttgcagtgactaactgtgccatgtcgtacaagggagcatggtggtataagaactgccaccggaccaacctcaatgggaagtacggggagtccaggcacagtcagggcatcaactggtaccattggaaaggccatgagttctccatcccctttgtggaaatgaagatgcgcccctacaaccaccgtctcatggcagggagaaaacggcagtccttacagttctgagcagtgggcggctgcaagccaaccaatattttctgtcatttgtttgtattttataatatgaaacaaggggggagggtaatagcaatgtgttttgcaacatattaagagtatgtgaaggaagcagggatgtcgcaggaatccgctggctaacatctgctcttggtttctgctgccctggagcctgaccctcagtctccattctccctcctacccaggcctcctcaaccttcacctcctttcccaccaaggaggagaagtaggaagttttcttaaagggccaattcaaagccaagtcgtggggtgcagattgttatggtgacaggcacacacatttttctacccttcttctgagatgtcctctgccttccaggtatttgtgattttgtcacagcctgacatggccaggttctcacactggcccagagaaaagagcctcagcaagagagttttgccaacaattccccttaaaaggaaacagatcaactacaccgcatcccaacaacccaggttcttttccttccttccttccttcctcccttccttctttcctgccttccc

SEQ ID NO: 2 - protein, 1358 aa, homo sapiens
MGADGETVVLKNMLIGVNLILLGSMIKPSECQLEVTTERVQRQSVEEEGGIANYNTSSKEQPVVFNHVYNINVPLDNLCSSGLEASAEQEVSAEDETLAEYMGQTSDHESQVTFTHRINFPKKACPCASSAQVLQELLSRIEMLEREVSVLRDQCNANCCQESAATGQLDYIPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGRCANGTCLCEEGYVGEDCGQRQCLNACSGRGQCEEGLCVCEEGYQGPDCSAVAPPEDLRVAGISDRSIELEWDGPMAVTEYVISYQPTALGGLQLQQRVPGDWSGVTITELEPGLTYNISVYAVISNILSLPITAKVATHLSTPQGLQFKTITETTVEVQWEPFSFSFDGWEISFIPKNNEGGVIAQVPSDVTSFNQTGLKPGEEYIVNVVALKEQARSPPTSASVSTVIDGPTQILVRDVSDTVAFVEWIPPRAKVDFILLKYGLVGGEGGRTTFRLQPPLSQYSVQALRPGSRYEVSVSAVRGTNESDSATTQFTTEIDAPKNLRVGSRTATSLDLEWDNSEAEVQEYKVVYSTLAGEQYHEVLVPRGIGPTTRATLTDLVPGTEYGVGISAVMNSQQSVPATMNARTELDSPRDLMVTASSETSISLIWTKASGPIDHYRITFTPSSGIASEVTVPKDRTSYTLTDLEPGAEYIISVTAERGRQQSLESTVDAFTGFRPISHLHFSHVTSSSVNITWSDPSPPADRLILNYSPRDEEEEMMEVSLDATKRHAVLMGLQPATEYIVNLVAVHGTVTSEPIVGSITTGIDPPKDITISNVTKDSVMVSWSPPVASFDYYRVSYRPTQVGRLDSSVVPNTVTEFTITRLNPATEYEISLNSVRGREESERICTLVHTAMDNPVDLIATNITPTEALLQWKAPVGEVENYVIVLTHFAVAGETILVDGVSEEFRLVDLLPSTHYTATMYATNGPLTSGTISTNFSTLLDPPANLTASEVTRQSALISWQPPRAEIENYVLTYKSTDGSRKELIVDAEDTWIRLEGLLENTDYTVLLQAAQDTTWSSITSTAFTTGGRVFPHPQDCAQHLMNGDTLSGVYPIFLNGELSQKLQVYCDMTTDGGGWIVFQRRQNGQTDFFRKWADYRVGFGNVEDEFWLGLDNIHRITSQGRYELRVDMRDGQEAAFASYDRFSVEDSRNLYKLRIGSYNGTAGDSLSYHQGRPFSTEDRDNDVAVTNCAMSYKGAWWYKNCHRTNLNGKYGESRHSQGINWYHWKGHEFSIPFVEMKMRPYNHRLMAGRKRQSLQF

SEQ ID NO: 3 - DNA, 23 bp, Artificial sequence
  Description of the artificial sequence primer
tccatgatcaagccttcagagtg

SEQ ID NO: 4 - DNA, 20 bp, Artificial sequence
  Description of the artificial sequence primer
aggggcaactgctgagcagt

SEQ ID NO: 5 - DNA, 22 bp, Artificial sequence
  Description of the artificial sequence primer
tcagcagttgcccctccagagg

SEQ ID NO: 6 - DNA, 21 bp, Artificial sequence
  Description of the artificial sequence primer
atgagggaacacccggcctcc

SEQ ID NO: 7 - DNA, 21 bp, Artificial sequence
  Description of the artificial sequence primer
atcacctccaccgctttcacc

SEQ ID NO: 8 - DNA, 24 bp, Artificial sequence
  Description of the artificial sequence primer
gaactgtaaggactgccgttttct

SEQ ID NO: 9 - DNA, 23 bp, Artificial sequence
  Description of the artificial sequence primer
tccatgatcaagccttcagagtg

SEQ ID NO: 10 - DNA, 21 bp, Artificial sequence
  Description of the artificial sequence primer
gccgtcaatgactgtggagac

SEQ ID NO: 11 - DNA, 22 bp, Artificial sequence
  Description of the artificial sequence primer
gccagcgtctccacagtcattg

SEQ ID NO: 12 - DNA, 22 bp, Artificial sequence
  Description of the artificial sequence primer
gttgtccatggctgtgtgcaca

SEQ ID NO: 13 - DNA, 20 bp, Artificial sequence
  Description of the artificial sequence primer
gtgcacacagccatggacaa

SEQ ID NO: 14 - DNA, 23 bp, Artificial sequence
  Description of the artificial sequence primer
gaactgtaaggactgccgttttc

(19) United States
(12) Patent Application Publication
(10) Pub. No.: US 2011/0159012 A1
(43) Pub. Date: Jun. 30, 2011

(54) METHOD FOR ISOLATING NEURAL CELLS USING TENASCIN-R COMPOUNDS

(75) Inventor: Penka Pesheva, Bonn (DE)

(73) Assignee: RHEINISCHE-FRIEDRICH-WILHELMS-UNIVERSITÄT BONN, BONN (DE)

(21) Appl. No.: 11/993,578

(22) PCT Filed: Dec. 16, 2005
(86) PCT No.: PCT/EP2005/056860

§ 371 Date: Feb. 23, 2010

(30) Foreign Application Priority Data
Dec. 20, 2004 (DE) 10 2004 062 420.8

(51) Int. Cl. A61K 39/395; C12N 5/079; C12Q 1/02; C07K 14/435; C12N 15/12; C12N 15/63; C12N 5/00; C12P 21/00; C07K 16/18; A61K 38/16; A61P 25/28

(52) U.S. Cl. 424/172.1; 435/325; 435/29; 530/300; 530/402; 536/23.4; 435/320.1; 435/69.1; 530/389.1; 530/388.1; 435/332; 514/17.7

(57) ABSTRACT

The invention relates to a process for isolating neural cells using tenascin-R compounds, tenascin-R fragments and tenascin-R fusion proteins that are particularly suitable for such process, the recombinant preparation of such tenascin-R compounds, and a kit for performing this process, and the use of the process for preparing highly pure neural cell populations. The invention further relates to antibodies suitable for the detection and isolation of tenascin-R compounds.

The invention relates to a process for isolating neural cells using tenascin-R compounds, tenascin-R fragments and tenascin-R fusion proteins that are particularly suitable for such process, the recombinant preparation of such tenascin-R compounds, and a kit for performing this process, and the use of the process for preparing highly pure neural cell populations. The invention further relates to antibodies suitable for the detection and isolation of tenascin-R compounds.

### BACKGROUND OF THE INVENTION

When cells come into contact with the surrounding extracellular environment, then cell may show a wide variety of responses that may range from rejection and avoidance of the environment on the one hand to stable cell adhesion on the other. The interplay between components of the extracellular environment, the extracellular matrix, on the one hand and receptors for such components present on the cell surface on the other hand represents the basis of many processes of development biology, which are characterized for a cell, inter alia, by proliferation, migration or differentiation behavior. Such cellular behavior leads to pattern formation on the organism level, or to new formation as a result of injury on the tissue or organ level (Boudreau, N. J., Jones, P. L., Biochem. J. 339: 481-488 (1999); Sobeih, M. M., Corfas, G., Int. J. Dev. Neurosci. 148: 971-84 (2002); Schmid, R. S., Anton, E. S., Cereb. Cortex. 13: 219-24 (2003)). In the central nervous system (CNS), the regulated expression of extracellular matrix components, such as chondroitin sulfate proteoglycans or proteins of the tenascin family, correlates with biological processes which include both the adhesion and migration of neurons, the navigation of axons, synapse formation and plasticity, the action of growth factors and cytokines as well as the survival of neurons and the structural organization of the extracellular matrix (Dow, K. E., Wang, W., Cell Mol. Life Sci. 54: 567-81 (1998); Wright, J. W. et al., Peptides 23: 221-46 (2002); Grimpe, B., Silver, J., Prog. Brain Res. 137: 333-49 (2002); Bosman, F. T., Stamenkovic, I., J. Pathol. 200: 423-8 (2003)).

Tenascin-R (TN-R) (formerly referred to as J1-160/180, janusin or restrictin) is a member of the tenascin family of extracellular matrix proteins that occurs exclusively in the CNS of vertebrates, where it is expressed by oligodendrocytes and some groups of neurons, i.e., motoneurons and interneurons, during later stages of the development and in the adult state (Pesheva, P., Probstmeier, R., Prog. Neurobiol. 61: 465-93 (2000); Scherberich, A. et al., J. Cell Sci. 117: 571-81 (2004)). The protein occurs in two molecular forms having molecular weights of 160 kD (TN-R 160) or 180 kD (TN-R 180). TN-R is found in the tissue primarily in association with oligodendrocytes, myelinized axons, perineuronal networks of motoneurons and interneurons, and in regions rich in dendrites and synapses.

TN-R is composed of four different domain structures (FIG. 1). The N terminus, whose sequence occurs only in tenascin proteins, contains a cysteine-rich segment (Cys-rich) and is followed by four and a half EGF-like segments (EGF-like) as well as 9 fibronectin type III (FN III) like domains (of which the 6th domain may be alternatively spliced). The C terminus of the TN-R protein is formed by a globular fibrinogen-like domain (ENG). Single TN-R polypeptide chains are connected through disulfide bridges at their N termini and thus form homotrimers (TN-R 180) or dimers (TN-R 160), the latter being formed by the proteolytic cleavage of TN-R 180 near the N terminus (Woodworth, A. et al., J. Biol. Chem. 279: 10413-21 (2004)).

The functional range of TN-R comprises the molecular control of neural cell adhesion, migration and differentiation (from the axon navigation of neurons forming processes to the maturation of myelin-forming oligodendrocytes) during normal development-biological processes as well as regenerative processes upon injury in the adult brain (Pesheva, P., Probstmeier, R., Prog. Neurobiol. 61: 465-93 (2000); Chiquet-Ehrismann, R., Int. J. Biochem. Cell. Biol. 36: 986-90 (2004)). Inter alia, TN-R acts as an adhesive or anti-adhesive molecule, as a differentiation factor for oligodendrocytes, or as a “stop molecule” for growing axons. These properties depend on the corresponding general conditions: the respective cell type, the presence of cellular receptors and signal cascades, the distribution in space and the posttranslational modification of the TN-R glycoprotein. Some of the cellular receptors of TN-R that have been identified (F3/F11, disialogangliosides, sulfatides) induce different cellular mechanisms of action which are important on the one hand in the pattern formation during ontogenesis and on the other hand in regenerative processes (Angelov, D. et al., J. Neurosci. 18: 6218-29 (1998); Probstmeier, R. et al., J. Neurosci. Res. 60: 21-36 (2000); Montag-Sallaz, M., Montag, D., Genes Brain Behay. 2: 20-31 (2003); Saghatelyan, A. et al., Nat. Neurosci. 7: 347-56 (2004); Brenneke, F. et al., Epilepsy Res. 58: 133-43 (2004)). The two essential effects of TN-R on the behavior of neural cells are the inhibition of cell adhesion and neurite growth on the one hand, and the promotion of oligodendrocyte adhesion and differentiation on the other. The latter is mediated by sulfatides and ultimately causes oligo-dendrocyte migration and myelinization/remyelinization. The former can occur either substrate-independently (mediated by F3/F11 and other, as yet unknown factors) or substrate/integrin-dependently (mediated by fibronectin and GD2/GD3). The inhibitory effect of TN-R ultimately has an influence on the neural cell migration, the space-coordinated axon growth and synaptogenesis, or it contributes to the prevention of axon regeneration and the adhesion of activated microglia in a TN-R-rich environment under injury conditions.

Some neural receptors and intracellular signal pathways that mediate the effect of TN-R in neuronal and glial cells are known, and molecular components involved in the expression of TN-R by oligodendrocytes and motoneurons could also be identified (Table 1; Pesheva, P. et al., Prog. Brain Res. 132: 103-14 (2001)).

TABLE 1
Cellular receptors and ligands of the extracellular matrix identified for
TN-R. In the right-hand half of the Table, the domains of TN-R relevant
for the respective interaction are stated; CS GAG: chondroitin sulfate
glycosaminoglycans; EGF-L: EGF-like segments and cystein-rich segment.

| | | Ligands of the | |
|---|---|---|---|
| Cellular | Tenascin-R | extracellular | Tenascin-R |
| receptors | binding domain | matrix | binding domain |
| F3/F11 | EGF-L, FN2-3 | Fibronectin | CS GAG, unknown |
| β2 subunit of | FN1-2, FN6-8 | Collagens | unknown |
| Na channels | | | |
| Neurofascin | FN2-5 | Tenascin-C | CS GAG, |
| | | | Ca²⁺-dependent |
| CALEB | FNG | Tenascin-R | unknown, |
| | | | cation²⁺-dependent |
| MAG | EGF-L, FNG | Lecticane | FN3-5, Ca²⁺-dependent |
| Sulfatides | unknown | Phosphacane | EGF-L, Ca²⁺-dependent |
| Gangliosides | unknown | | |

As TN-R is an extracellular matrix protein constituted of several domains, an attribution of the distinct biological functions to distinct domain regions and/or distinct glycostructures of TN-R suggests itself (FIG. 1), It is known that TN-R proteins purified from adult rodent brain promote the adhesion and process formation of O4/sulfatide-positive oligodendrocytes isolated from early postnatal brain (FIG. 3; Pesheva, P. et al., J. Neurosci. 17: 4642-51 (1997)). These processes are mediated by sulfatides, a group of glycolipids occurring in the cell membrane of oligodendrocytes. An important consequence of the interaction of TN-R with sulfatide-expressing oligodendrocytes is a stimulation of the maturation of these cells; i.e., the increased expression of myelin-specific proteins and glycolipids, which suggests a sulfatide-mediated mode of action of the differentiation potential of TN-R on oligodendrocytes (FIG. 4; Pesheva, P. et al., J. Neurosci. 17: 4642-51 (1997)).

EP 0 759 987, U.S. Pat. No. 5,635,360, U.S. Pat. No. 5,681,931 and U.S. Pat. No. 5,591,583 describe human TN-R and its immunological detection using antibodies directed against a protein fragment that corresponds to the nucleotides 2686-3165 of the cDNA sequence and thus to the FN III domains 6 and 7 of human TN-R.

The earliest stages of developing glia cells in mammals are found in ventral regions of the neural tube in the spinal cord, and in ventricular zones of the fore-brain in the brain. In these regions, first precursor cells of oligodendrocytes characterized by a simple morphology and the expression of the disialogan-glioside GD3 and/or of O4 antigens proliferate and migrate in the following time into regions of the later formed white substance (Miller, R. H. Prog. Neurobiol. 67: 451-67 (2002); Noble, M. et al., Dev. Biol. 265: 33-52 (2004); Liu, Y. Rao, M. S., Biol. Cell. 96: 279-90 (2004)). There, the cells become postmitotic and differentiate into mature oligodendrocytes with a complex morphology. This maturation process correlates with the expression of myelin-specific lipids (sulfatides and galactocerebrosides) and proteins (MBP, MAG and PLP). The formation, the survival and the differentiation of oligodendrocytes in myelin-forming cells are regulated through various growth factors (bFGF, PDGF, CNTF, IGF-1, NT-3), hormones and extracellular matrix molecules (thyroid hormones, retinolic acid, TN-R) (Dubois-Dalcq, M., Murray, K., Pathol. Biol. (Paris) 48: 80-6 (2000); Kagawa, T. et al., Microsc. Res. Tech. 52: 740-5 (2001); Noble, M. et al., Dev. Neurosci. 25: 217-33 (2003)).

Since especially for glial cells (such as oligodendrocytes and neural stem cells) no adequate model systems in the form of cell lines exist, relatively time-consuming and low-efficient enrichment processes of primary cells have had to be recurred to to date in their recovery both in basic research and within the scope of potential diagnostic/therapeutic fields of application. In addition, these processes often enable only the preparation of enriched mixed cell populations. The previous processes for isolating defined cell populations utilize different techniques, such as density gradient centrifugations or immunological processes (Fluorescence-activated cell sorting, biomagnetic cell sorting, antibody- and complement-mediated cell killing, antibody panning) (Luxembourg, A. T. et al., Nat. Biotechnol. 16: 281-5 (1998); Uchida, N. et al., Proc. Natl. Acad. Sci. 97: 14720-5 (2000); Nistri, S. et al., Biol. Proced. Online 4: 32-37 (2002); Nunes, M. C. et al., Nat. Med. 9: 439-47 (2003); Vroemen, M., Weidner, N., J. Neurosci. Methods 124: 135-43 (2003)). Such methods are not very efficient and result in an incomplete enrichment (by density gradient centrifugations, for example, enrichments of distinct cell populations are only possible), or they are time-intensive and/or accompanied by high cell losses (as in immunological processes). This applies, in particular, to oligodendrocytes which to date have been obtained by several weeks of cultivation of mixed glial cultures (McCarthy, K. D., DeVellis, J., J. Cell Biol. 85: 890-902 (1980); Kramer, E. M. et al., J. Biol. Chem. 274: 29042-9 (1999); Testal, F. D. et al., J. Neurosci. Res. 75: 66-74 (2004)) or by several selection steps by means of fluorescence-activated/biomagnetic cell sorting or antibody panning (Scarlato, M. et al., J. Neurosci. Res. 59: 430-5 (2000); Tang, D. G. et al., J. Cell Biol. 148: 971-84 (2000); Diers-Fenger, M. et al., Glia 34: 213-28 (2001); Crang, A. J. et al., Eur. J. Neurosci. 20: 1445-60 (2004)).

### SUMMARY OF THE INVENTION

The isolation of defined cell populations from primary tissues, especially those of neural origin, is laborious and time-intensive by the previously known methods, and as a result, little enriched cell mixed populations are frequently obtained. Now, it has been found that purified TN-R proteins support the stable adhesion of oligodendrocytes in different stages of maturity and have an anti-adhesive effect on neuronal and microglial cells. This enables the isolation and purification of defined cell populations from neural primary tissue, especially for the direct selective purification of oligodendrocytes from mixed neural cell populations.

A process is provided that enables the isolation of a highly pure defined cell population, especially an oligodendrocyte population, from primary tissue of neural origin in a single purification step.

In detail, the invention relates to:
(1) a process for the isolation and purification of neural cells from neural primary tissue of vertebrates, comprising the selection of the cells from a single cell suspension by means of a probe containing tenascin-R (also referred to as “tenascin-R probe” in the following), which includes tenascin-R compounds selected from native tenascin-R (briefly referred to as “TN-R” in the following) as well as homologues and fragments thereof and fusion proteins of such compounds;
(2) a preferred embodiment of the process (1) as defined above, wherein
(i) said native tenascin-R is human tenascin-R and/or has the amino acid sequence of SEQ ID No. 1 or is a substitution, deletion and/or addition mutant thereof; and/or
(ii) said tenascin-R fragment comprises the C terminus of native tenascin-R or a substitution, deletion and/or addition mutant thereof, especially the region encoded by the nucleotides 3940-4155 of SEQ ID No. 1, especially one of those regions encoded by the nucleotides 2926-4155, 3439-4155, 3487-4155 or 3940-4155 of human TN-R of SEQ ID No. 1; and/or
(iii) said tenascin-R fragment comprises the amino acid residues 1287 to 1358 of SEQ ID No. 2, preferably one or more of the partial sequences of human TN-R selected from the amino acids 1287-1358, 1120-1358, 1136-1358 or 949-1358 in SEQ ID No. 2 or a substitution, deletion and/or addition mutant thereof; and/or
(iv) said tenascin-R fusion protein includes a tenascin-R component comprising native tenascin-R, a tenascin-R fragment or a tenascin-R mutant, especially as described above under (i) to (iii), and a functional component comprising further functional peptides or proteins; or

is composed of two or more, preferably two or three, functional tenascin-R components as defined above;
(3) a preferred embodiment of the process (1) or (2) as defined above, wherein
(i) said tenascin-R probe is bound to a support material by non-covalent interactions (such as interaction with TN-R-specific antibodies etc,) or by another adequate coupling technique which does not change the specificity of the tenascin-R probe (such as covalent cross-linking etc.); and/or
(ii) said single cell suspension is contacted with said tenascin-R probe so that tenascin-R-binding cells present in said single cell suspension become bound to said probe; and/or
(iii) isolation of these cells from the cell culture is effected by specific binding of neural stem cells from said single cell suspension to said tenascin-R probe, the unbound cells are removed, and optionally the cells bound to the support material through said tenascin-R probe are subsequently detached from the support material by trypsinization, incubation with Accutase® or another adequate method; and/or
(iv) the bound cells are detected by immunological methods; and/or
(v) the process is effected in vitro;
(4) a tenascin-R fragment or tenascin-R fusion protein as defined above under (1) or (2) and preferably having an amino acid sequence selected from amino acids 1287-1358, 1120-1358, 1136-1358 or 949-1358 in SEQ ID No. 2;
(5) a DNA which codes for a tenascin-R fragment or TN-R fusion protein according to (4);
(6) a vector which comprises a DNA according to (5);
(7) a host organism transformed/transfected with a vector according to (6) and/or having a DNA according to (5);
(8) a process for preparing a tenascin-R fragment or TN-R fusion protein according to (4), comprising the step of culturing said host organism according to (7);
(9) an antibody obtainable by the immunization of a suitable host organism with tenascin-R from at least two different species, especially with TN-R from at least two different vertebrates, and/or which binds to TN-R from at least two different species, especially from at least two different vertebrates, i.e., shows cross-reactivity with different vertebrate TN-R;
(10) a preferred embodiment of the antibody according to (9), wherein said antibody is a monoclonal antibody;
(11) a cell line which produces said antibody according to (10);
(12) a kit for the isolation and purification of neural cells, especially of oligodendrocytes, according to one or more of processes (1) and (2), especially containing
(i) a tenascin-R probe as defined in (1) or (2); and/or
(ii) a vector which codes for such a tenascin-R probe; and/or
(iii) a stock culture of a cell line which is suitable for expressing said tenascin-R probe as defined in (1) or (2), preferably for expressing it recombinantly;
(13) the use of said tenascin-R probe according to (1) or (2) for obtaining neural cells, especially oligodendrocytes, for growing differentiated cells, especially neural cells, in neurobiological and cell-physiological examinations, in biological and clinical research and for diagnostic and therapeutic processes in vitro and in vivo, especially for the preparation of a medicament for cell therapy and for the therapy of neurodegenerative diseases accompanied by a loss of oligodendrocytes or myelin, especially multiple sclerosis and periventricular leukomalacia (PVL); and
(14) the use of said antibody according to (9) or (10)
(i) for the immunochemical detection of TN-R;
(ii) for the inhibition of the effect of TN-R;
(iii) for influencing the neural development; and
(iv) for preparing medicaments for the therapy and prophylaxis of traumatic neural lesions and medicaments for the selective influencing of neural development;
(15) a process for the therapy and prophylaxis of traumatic nerve lesions or for selectively influencing the neural development, comprising the step of administering a pharmacologically sufficient amount of the antibody according to (9) or (10) to a human or animal patient in need of such treatment;
(16) a process for cell therapy or for the therapy of neurodegenerative diseases accompanied by a loss of oligodendrocytes or myelin, especially multiple sclerosis and periventricular leukomalacia (PVL), comprising the step of administering a tenascin-R probe as defined in (1) or (2), preferably a tenascin-R fragment or tenascin-R fusion protein as defined in (4), to a human or animal patient; and
(17) a process for preparing oligodendrocytes from isolated stem cells in vitro by incubating the stem cells in the presence of a tenascin-R probe as defined in (1) or (2), preferably a tenascin-R fragment or tenascin-R fusion protein as defined in (4).

### BRIEF DESCRIPTION OF THE FIGURES

FIG. 1: Structure of TN-R. The position of the alternatively spliced FN III like domain is shown as R1. The insert shows electron micrographs of rotary-shadowed TN-R molecules purified from mouse brain. Single polypeptide chains are interconnected at their N-terminal ends through disulfide bridges, which results in the formation of dimers (TN-R 160) or trimers (TN-R 180).

FIG. 2: CLUSTAL W (1.82) alignment of the known amino acid sequences of tenascin-R from different vertebrates. “*” designates identical amino acids; designates a conservative amino acid exchange; “.” designates a semiconservative amino acid exchange. The high phylogenetic conservation of the C terminus containing the FNG domain can be clearly seen.

FIG. 3: Influence of different polar glycolipids and O4 antibodies on the cell adhesion by TN-R. TN-R substrates were preincubated in the absence (−GL) or presence of sulfatides (+Sulf), galactocerebrosides (+GalC), monosialogan-gliosides (+GM1) and sphingosine (+Sulf). Oligodendrocytes (OL, left side of the Figure) or erythrocytes (RBC, right side of the Figure) were sown in the presence or absence of O4 antibodies (+O4 Ab) onto the correspondingly treated substrates. The number of adherent cells after an incubation time of one hour on untreated TN-R substrates was set to 100%.

FIG. 4: Influence on the differentiation of oligodendrocytes by TN-R. Oligodendrocytes purified from early postnatal mouse brain were sown on PLL (poly-L-lysine) substrates in the absence (−TN-R) or presence (+TN-R) of substrate-bound TN-R proteins (purified from human or rat brain). The expression of myelin-specific proteins (MBP) was detected after 2 days in culture by means of indirect immunofluorescence staining.

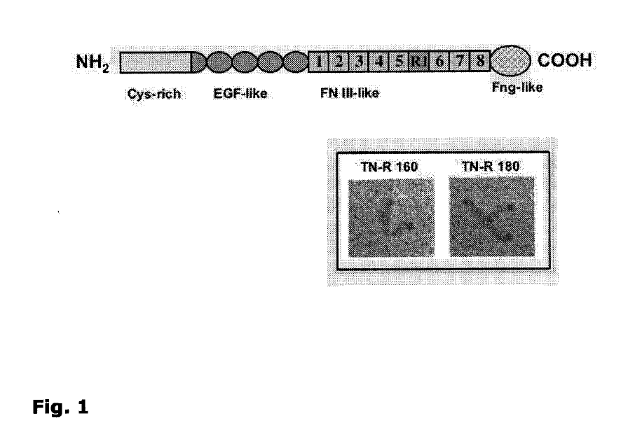

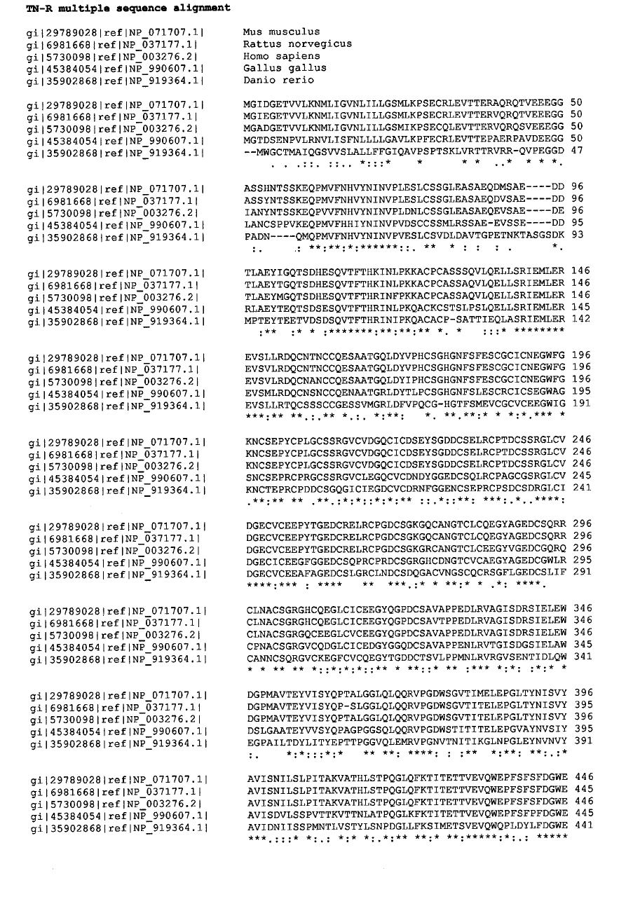

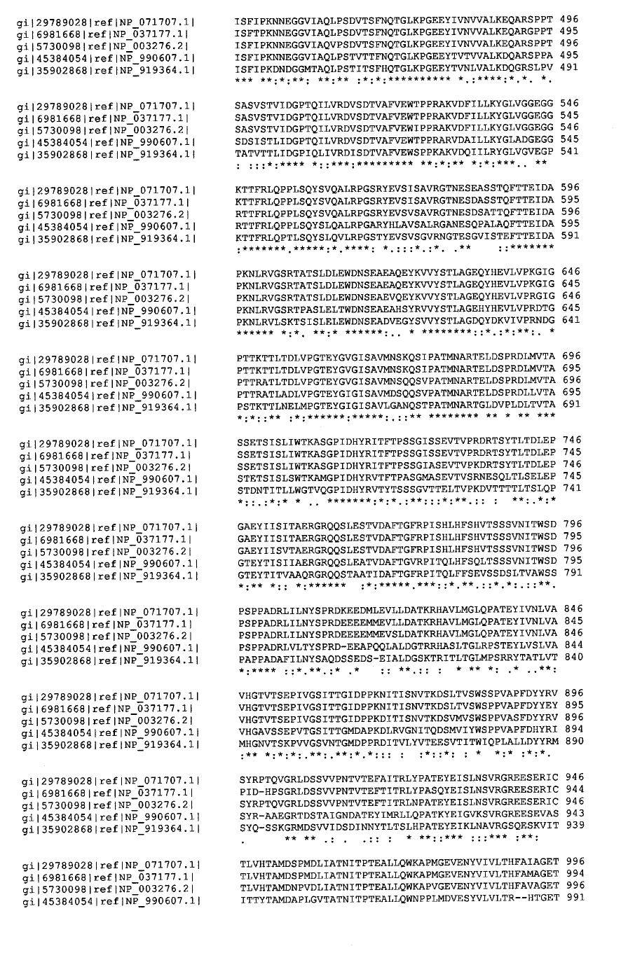

FIG. 5: Characterization of the specificity and functional activity of the monoclonal antibodies R1, R2, R4, R5 and R6.
A) ELISA assay for the analysis of the cross-reactivities of R4 and R6 with TN-R proteins of different vertebrate classes. Microtitration plates were coated with brain extracts of different vertebrates (40 μg/ml), and the binding of antibodies R4 and R6 (after 2 hours of incubation at 37° C.) was detected with peroxidase-coupled anti-mouse IgG antibodies. The maximum binding for the respective antibody was set at 100%.
B) Western blot analysis of brain extracts of different vertebrates with R6 antibody. Brain extracts (50 μg of total protein/well) from shark (Squalus), goldfish (Carassius), salamander (Salamandra), grass snake (Natrix), Greek tortoise (Testudo), pigeon (Columba), hedgehog (Erinaceus), mouse (Mus) and human (Homo) were separated by gel electrophoresis, transferred to nitrocellulose filters and incubated with R6 antibodies. Immunoreactive protein bands were detected by incubation with peroxidase-coupled anti-mouse IgG antibodies. Immunoaffinity-purified TN-R from mouse brain (m. TN-R) with the characteristic protein bands at 160 and 180 kD served as a reference.
C) Influence of R4 and R6 antibodies on the TN-R mediated inhibition of neural cell adhesion and neurite formation (Example 6). Hindbrain neurons from 8 days old mice were sown onto mixed substrates (in a ratio of 1:1) consisting of laminin and bovine serum albumin (BSA, control substrate) or laminin and human TN-R (h. TN-R). Before plating the cells, the substrates were incubated in the absence (−Ab) or presence of R4 or R6 antibodies (+R4/R6). The cell adhesion and neurite formation were evaluated by optical microscopy 2 days after culture start.
D) Long term adhesion of mouse hindbrain neurons on poly-L-lysine (PLL) TN-R substrate under the influence of the monoclonal antibodies. A substrate with BSA (control) or TN-R from humans was preincubated in the absence (−Ab) or presence of TN-R antibodies, followed by plating the neurons. The number of neurons adhering to the control substrate after 24 h was set at 100%.
E) Western blot of R4 with different tissues and tenascins. The tissues tested included brain, heart, liver, kidney and lung of adult mice, skin and skin fibroblast conditioned medium (CM) of neonatal mice, TN-R 160 from adult mouse brain and TN-C (br. TN-C) from early postnatal mouse brain. pTN-C Ab: polyclonal antibody against TN-C; R4 mAb: monoclonal antibody against TN-R.

FIG. 6: TN-R proteins purified by immunoaffinity from adult brain of different vertebrate species. Lane 1 (shark), lane 2 (carp), lane 3 (chicken), lane 4 (mouse), lane 5 (rat), lane 6 (human). TN-R can be detected as a polypeptide with the main forms of 220 kD (in shark brain), 170 kD (in carp brain) or 160 kD and 180 kD (in the brain of higher vertebrates).

FIG. 7: Selection of oligodendrocytes from single cell suspensions (from brain tissue of 2 (P2), 5 (P5) and 8 (P8) days old mice) for substrate-bound TN-R proteins isolated from the brain of different vertebrates (carp, chicken (ch), rat, human) of different vertebrate classes (fish, birds, mammals). Cells adhering to these substrates were stained with toluidine blue after one day of culture (upper lines). Adherent cells could be identified as oligodendrocytes by means of indirect immunofluorescence staining with antibodies against myelin-specific glycolipids (GaIC) after 2 (P8) to 5 days (P2) in culture (lower line; the image section does not correspond to the image section of the upper lines). 99±1% of the isolated cells were GaIC-positive.

FIG. 8: Ability of the TN-R from different species to induce oligodendrocyte differentiation.
A) MBP expression by mouse oligodendrocytes after 48 h of culture on PLL-TN-R substrate.
B) Autocrine regulation of TN-R secretion by cultured mouse oligodendrocytes. The TN-R secretion by oligodendrocytes on PLL-TN-R was compared with that of oligodendrocytes on PLL-BSA and expressed as a multiple of protein secretion (protein increase),

### Sequence Listing—Free Text

| SEQ ID No. | Description |
|---|---|
| 1 and 2 | TN-R |
| 3-14 | primer |

### DETAILED DESCRIPTION OF INVENTION

The invention relates to a process for isolating highly pure cell populations from neural primary tissue in a one-step process by means of native tenascin-R proteins or TN-R fragments of vertebrates, preferably fish, amphibians, reptiles, birds and mammals, more preferably shark, carp, chicken, rodents including mouse and rat, cattle, pig and human, even more preferably rodents and humans, by selective substrate adhesion.

Phylogenetic studies relating to the function of tenascin-R in the nerve system of vertebrates show the support of adhesion and process formation in oligodendro-cytes as a function of tenascin-R proteins that is highly conserved in evolution (FIGS. 3 and 7). The present invention shows that this property of the total tenascin-R protein can also be localized on distinct recombinant fragments of human tenascin-R protein.

In the context of the present invention, “tenascin-R probe” means a protein that may contain native tenascin-R recognized and bound by oligodendrocytes in vivo and in vitro, and/or further tenascin-R compounds including tenascin-R homologues and tenascin-R fragments that are also bound with high sensitivity and specificity in vivo and in vitro by oligodendrocytes and/or other neural cells from primary tissue, wherein “tenascin-R compounds” has the meaning as defined elsewhere. This also includes fusion proteins of several tenascin-R compounds, preferably from two or three tenascin-R compounds. The components of these fusion proteins can be interconnected directly or through a (flexible) linker peptide. Fusion proteins of TN-R fragments are preferred. More preferably, the tenascin-R probe contains human tenascin-R and its homologues and fragments, even more preferably the partial sequence of human TN-R comprising the amino acid residues 1287-1358 of SEQ ID No. 2. In addition to its TN-R portions, the tenascin-R probe may also have non-TN-R portions which ensure the stability of the TN-R portion and/or maintain or increase the immobilizability and coupling ability to other molecules. In particular, a tenascin-R probe according to the invention is prepared recombinantly.

According to the invention, “tenascin-R compounds” are proteins or peptides which are either native TN-R, substantially identical with native TN-R, fragments of native TN-R or their substantially identical homologues.

“Primary tissue” is a biological tissue which is taken directly from an organism and used without further modifying its genetic material. “Primary cells” are cells originating from primary tissue. Primary cells are advantageous over cell lines because they are closest to the cells in the intact organism structurally and functionally due to their origin.

In the case of a protein, “isolated” means that it has been separated or purified from other proteins with which it is normally associated in the organism in which it naturally occurs. This includes biochemically purified proteins, recombinantly prepared proteins and proteins synthesized on a chemical route. The definition also applies, mutatis mutandis, to nucleic acids, especially DNA, and peptides.

“Native” is used interchangeably with “natural” or “naturally occurring”.

For the “recombinant preparation” according to the invention, methods usual in the art for the recombinant preparation of eukaryotic proteins are used, especially the expression as a fusion protein in eukaryotic cells, more preferably the expression as a fusion protein having a polyhistidine tail and/or Xpress tag. Further preferred is the use of DNA coding for the corresponding protein.

Within the scope of the present invention, nucleic acid sequences, especially DNA sequences coding for proteins or peptides according to the invention, are either identical or substantially identical with the native sequence or its underlying artificial sequence according to the invention. If a specific nucleic acid sequence is mentioned within the scope of the present invention, it includes this sequence itself and the sequences substantially identical therewith. “Substantially identical” means that only an exchange of bases in the sequence within the scope of the degenerate nucleic acid code has been effected, i.e., that the codons within coding sequences of the substantially identical nucleic acid are changed thereby as compared to the original molecule merely in a way that does not lead to a change of the amino acid sequence of the translational product (usually exchange of the codon by another codon of its codon family). Within the scope of the present invention, sequences which are mentioned in the Sequence Listing and their fragments according to the invention are preferred.

Protein sequences and peptide sequences can be modified within the scope of the present invention by the substitution of amino acids. Preferred are those substitutions in which the function and/or conformation of the protein or peptide is retained, more preferably those substitution in which one or more amino acids are replaced by amino acids which have similar chemical properties, e.g., alanine for valine (“conservative amino acid exchange”). The proportion of substituted amino acids as compared to the native protein or, if it is not a native protein, to the starting sequence is preferably 0-30% (based on the number of amino acids in the sequence), more preferably 0-15%, even more preferably 0-5%.

Nucleic acid sequences and amino acid sequences can be employed as full length sequences or as addition or deletion products of such full length sequences for performing the invention. In the amino acid sequences, the addition products also include fusion proteins and additionally amino acid sequences formed by the addition of 1400, preferably 1-30, more preferably 1-10 amino acids. The added amino acids may be inserted or added singly or in contiguous segments of 2 or more interconnected amino acids. The addition may be effected at the N or C terminus or within the original sequence. Several additions in one sequence are allowed, wherein a single addition is preferred, more preferably an addition at the C or N terminus.

The deletion products of the full length amino acid sequences are formed, unless stated otherwise for specific sequences, by the deletion of 1-100, preferably 1-20, more preferably 1-10 amino acids. The deleted amino acids may be removed singly or in contiguous segments of 2 or more interconnected amino acids. The deletion may occur at the N or C terminus or within the original sequence. Several deletions in one sequence are allowed, wherein a single deletion is preferred, more preferably a deletion at the C or N terminus.

The allowable deletions and additions in the nucleic acid sequences according to the invention have the extent and nature that correspond to the allowable amino acid deletions or additions. Apart from the deletion and addition of entire codons, the addition or deletion of single bases or pairs of bases is also possible.

A fragment of a nucleic acid or protein is a part of its sequence that is shorter than the full length, but still contains a minimum sequence segment required for hybridization or specific binding. In the case of a nucleic acid, this sequence segment is still capable of hybridizing with the native nucleic acid under stringent conditions and preferably comprises at least 15 nucleotides, more preferably at least 25 nucleotides. In the case of a peptide, this sequence segment is sufficient to enable the binding of an antibody specific for a segment of the native protein or of a cell that binds to TN-R or a TN-R fragment. The peptide length is preferably at least 5 amino acids, more preferably at least 10 amino acids, even more preferably at least 20 amino acids.

A “fusion protein” in the context of the present invention comprises at least one tenascin-R compound according to the invention that is linked to at least one second protein or peptide. Such second protein or peptide is preferably a selection or marker protein, a protein that serves for the binding of the fusion protein to a surface, or a tenascin-R compound. Preferred are fusion proteins of native tenascin-R and further functional proteins and peptides, and fusion proteins of two or more, more preferably two or three, tenascin-R fragments. The nucleic acid sequences coding for the individual parts of the fusion protein in a vector or transformed host organism are connected with one another in a way that allows the expression under the control of a single promoter, The amino acid sequences of the individual functional parts of the fusion protein are linked to one another either directly or through a linker. The linker has a length of 1-30 amino acids, preferably 10-20 amino acids.

“Neurodegenerative diseases” are diseases of the nerve system associated with the dying of neuronal and/or macroglial cells due to impairment of their functional integrity. Such cell damages and losses lead to failure or impairment of the functions of the affected regions of the nerve system and/or the body parts controlled by these regions.

The cells isolated by the process according to embodiment (1) and (2) are preferably glial cells, more preferably oligodendrocytes.

The tenascin-R probe for use in embodiment (1) comprises either a native TN-R protein or a TN-R protein fragment. The native TN-R preferably originates from brain tissue of fish (shark, carp, goldfish, trout etc.), amphibians (salamander, frog etc.), reptiles (Greek tortoise, grass snake etc.), birds (chicken, pigeon etc.) and mammals (hedgehog, rabbit, rodent, pig, cattle, human), especially mammals, more especially rodent, pig, cattle or human. The native TN-R protein is preferably prepared recombinantly. The TN-R protein fragments are preferably prepared recombinantly, and/or are human TN-R fragments. In the latter case, a preferred source of the corresponding DNA sequence is the human neuroblastoma cell line SH-SY5Y. The expression of the fragments is preferably effected after transformation of human Flp-In 293 cells (Invitrogen) with the corresponding DNA sequences (Example 2).

In embodiment (1), the TN-R probe may additionally contain further functional protein or peptide sequences and/or be coupled to a support.

A preferred aspect of embodiment (1) and (2) is the use of the C terminus of TN-R in the tenascin-R probe, especially those regions which represent the FN III domains 7 and 8 as well as the FNG domain. These regions may be used either singly or in a form linked to one another, wherein the presence of the FNG domain in the probe is preferred. Particularly preferred is the use of the region of human TN-R that comprises the FNG domain, i.e., is coded by the nucleotides 3940-4155 of SEQ ID No. 1; more preferably the use of those regions of human TN-R which are coded by by 2926-4155 (FN III 7,8 +FNG domain; Ex. 2: H-TNR-6), and/or by by 3439-4155 (FNG domain; Ex. 2: H-TNR-3), by 3940-4155 and/or by 3487-4155 in SEQ ID No. 1.

Thus, DNA fragments of embodiment (5) preferably comprise by 3940-4155, 2926-4155, by 3487-4155 and/or by 3439-4155 of SEQ ID No. 1.

Thus, preferred tenascin-R fragments for use in a tenascin-R probe are fragments that contain the C terminus of TN-R. The C terminus of the TN-R protein forms a fibrinogen-like domain in which four cysteine residues including the surrounding four to five amino acids are highly conserved in terms of their position and composition in higher vertebrates. This region, preferably the region which comprises the amino acids 1287-1358 in human TN-R, more preferably the region which corresponds to the amino acids 1287-1358, 1120-1358, 1136-1358 (Carnemolla, B. et al., J. Biol. Chem. 271: 8157-8160 (1996)) or 949-1358 in SEQ ID No. 2, is preferred for use as a tenascin-R fragment in the TN-R probe for the adsorption of oligodendrocytes. In addition, this region represents the preferred sequence of the tenascin-R fragments according to embodiment (4). Particularly preferred as TN-R fragment are peptides having the sequence of amino acid residues 9494358 or 1120-1358 of SEQ ID No. 1.

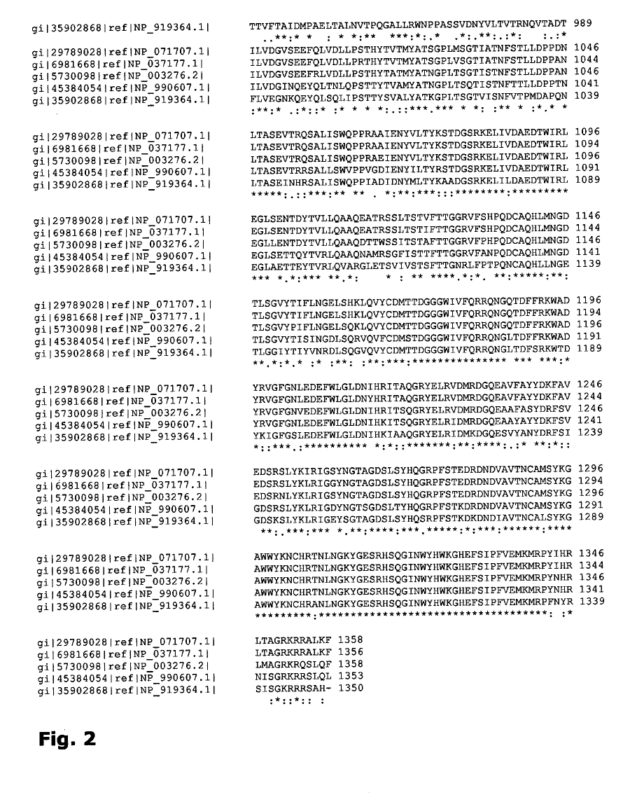

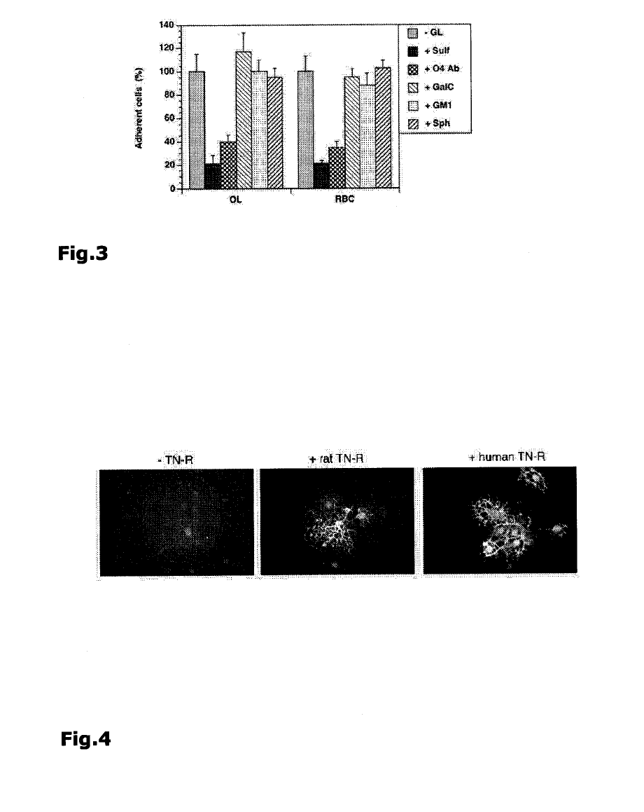

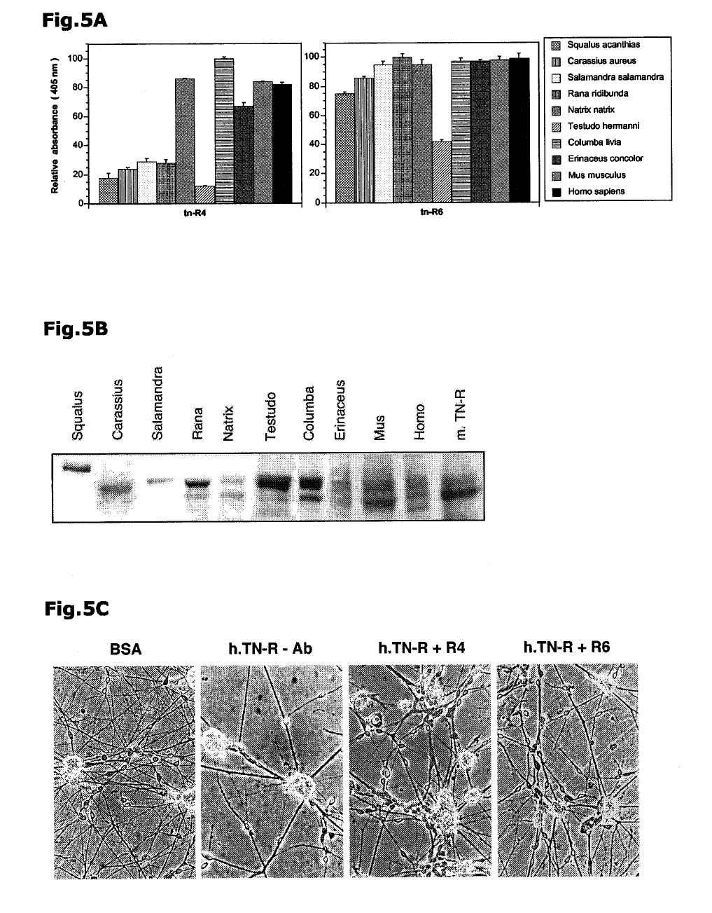

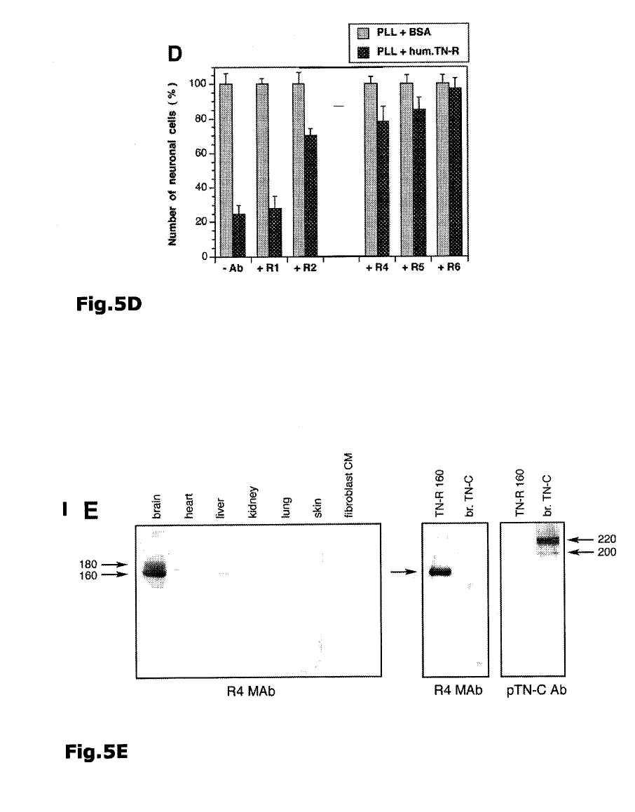

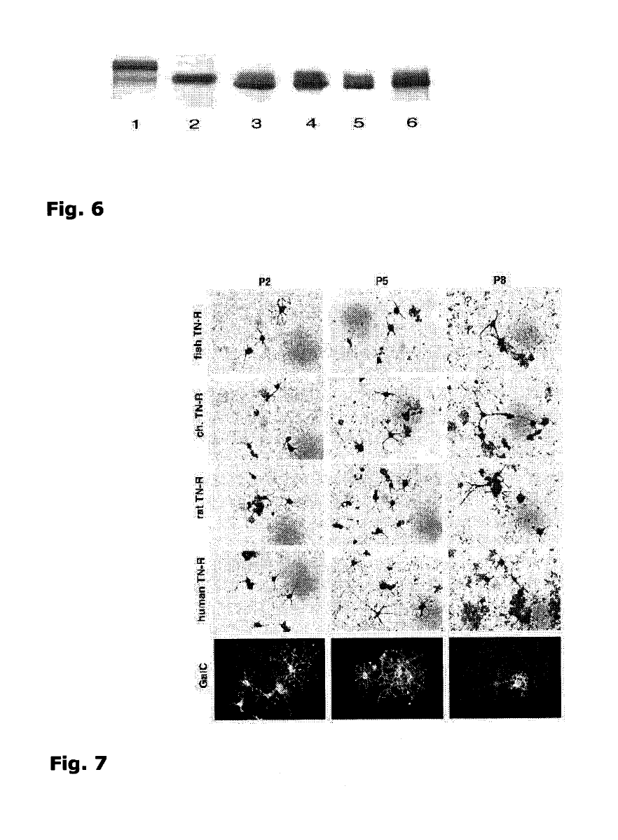

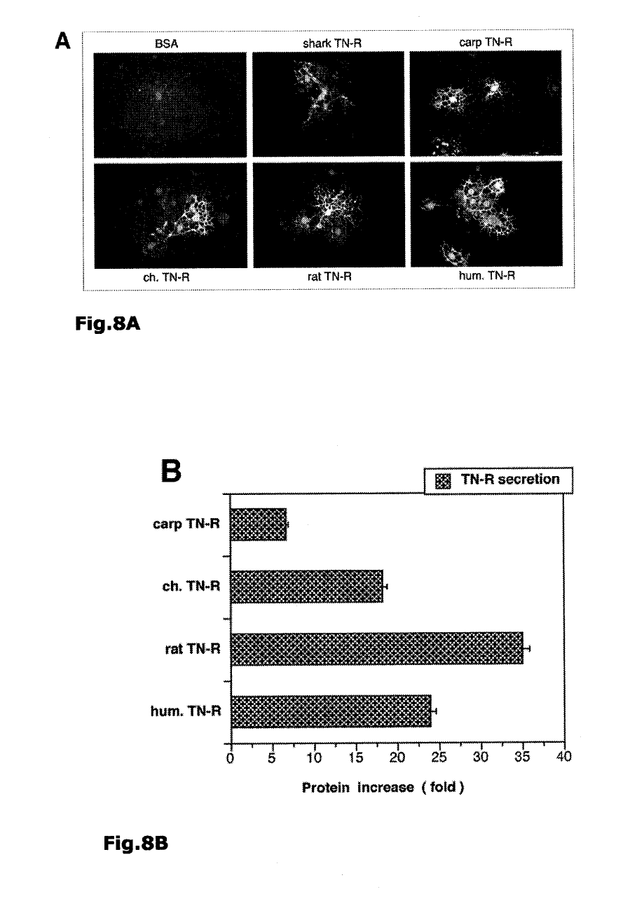

The phylogenetically conserved property of TN-R proteins, i.e., being able to act as an adhesive substrate for oligodendrocytes, is reflected by a high conservation of the amino acid sequence between different vertebrate species on the molecular level: the human TN-R sequence shows homologies of 93% (with rat), 75% (with chicken) and 60% (with zebra fish) (FIG. 2). This property is utilized by the present application.

One aspect of embodiment (1) is the preparation of the tenascin-R probe by isolating tenascin R from natural sources (cf. Ex. 1) or as a recombinant native protein. Preparation by recombinant methods is preferred.

The isolation of TN-R from natural sources is preferably effected by known chromatographic and/or immunological methods for protein purification, especially affinity chromatography on TN-R antibodies (Example 1). As a source of TN-R, tissues and single cell suspensions of higher and lower vertebrates may be used.

Recombinant methods for the preparation of the TN-R probe include the usual known methods for the transformation of prokaryotes and eukaryotes (as described, e.g., in G. Schrimpf (Ed.), Gentechnische Methoden, 3rd edition, Spektrum Akademischer Verlag (2002); Smith, C., The Scientist 12(3): 18 (1998); Unger, T., The Scientist 11(17): 20 (1997)). Suitable methods for the preparation of recombinant proteins or protein fragments comprise transfection or transformation methods based on different expression systems/vectors for prokaryotic (especially E. coli) and eukaryotic cells (yeast, fungi, insect and mammal cells). In order to produce functionally active recombinant proteins (i.e., those which are the most similar to the native protein after their folding/conformation and glycosylation), mammal cells are preferred as producers. The different expression vectors for mammal cells are mainly distinguished in the type of promoter (SV40, CMV, human EFlaipha, MMTV-LTR, MSV-LTR, RSV-LTR, etc.), kind of expression (transient, constitutive, inducible), induction mechanism, selective marker (antibiotic or drug resistance and/or co-expression of easily detected proteins) and elements for the subcellular targeting of the gene product (mitochondria, nucleus, secretion). Eukaryotic expression systems that express the foreign gene constitutively are preferred as producers of recombinant proteins/protein fragments.

In a preferred embodiment of (2) for the expression of human recombinant protein fragments according to the invention, defined PCR fragments of human TN-R are cloned by means of the TOPO TA cloning system (Invitrogen) into the pcsecTag/FRT/V5-His-TOPO vector (Invitrogen), which allows the constitutive expression and secretion of the desired protein fragment provided with a 6×His peptide at the C terminus in human Flp-In 293 cells (Invitrogen). In Flp-In cells, the plasmid pFRT/lacZeo (Invitrogen) is stably integrated, and the FRT region is specifically recognized by Hp recombinase. When the Hp 293 cells are simultaneously transfected with the pOG44 plasmid, which enables the expression of Flp recombinase, and the pcsecTag/FRT/V5-His-TOPO vector, which bears the base sequence of the desired protein fragment, an incorporation of the portions of the pcsecTag/FRT/V5-His-TOPO vector necessary for the preparation of a secreted protein fragment occurs at the FRT region. This enables the secretion of polyHis-bearing protein fragments into the cell culture supernatant and their purification by nickel chelate chromatography from collected cell culture supernatants.

Another aspect of embodiment (1) is the preparation of the TN-R fragments by chemical synthesis by the fragmentation of isolated TN-R or recombinantly, preferably recombinantly, according to embodiment (8). Suitable recombinant methods include the usual known methods for the transformation of prokaryotes and eukaryotes (as described, e.g., in G. Schrimpf (Ed.), Gentechnische Methoden, 3rd edition, Spektrum Akademischer Verlag (2002); Smith, C., The Scientist 12(3): 18 (1998); Unger, T., The Scientist 11(17): 20 (1997)). For this purpose, a host organism according to embodiment (7) can be used that is transformed or transfected with a vector which comprises the above defined DNA sequences coding for TN-R or TN-R fusion protein. In addition to the mentioned DNA sequences, such a vector may also contain functional sequences adapted to the host organism, such as promoters, leader sequences etc. For the chemical preparation of TN-R fragments and for the fragmentation of isolated TN-R, methods known in the art can be used, such as solid-phase peptide synthesis and enzymatic or mechanic fragmentation methods.

Another preferred aspect of embodiment (1) relates to a fusion protein comprising a tenascin-R component selected from native tenascin-R or tenascin-R fragments and fusion proteins of two or more tenascin-R fragments, and a functional component which comprises functional peptide or protein sequences. The components can be connected with one another directly or by means of a (flexible) linker peptide. Another aspect relates to the combination of two or more of the above defined tenascin-R components, especially tenascin-R fragments, in a way that does not correspond to the native amino acid sequence to form a tenascin-R probe according to the invention. The linking may also be direct or by means of a linker peptide. For the synthesis, the above mentioned vector systems can be used, wherein those cDNA sequences of parts of the TN-R sequence that are not adjacent in space are linked to one another through terminal restriction enzyme cleavage sites according to usual technical methods.

The tenascin-R probe according to embodiments (1) to (4) preferably comprises the partial sequence of human TN-R that comprises the amino acid residues 1287-1358, especially the region which corresponds to the amino acid residues 1287-1358, 1120-1358, 1136-1358 or 949-1358 in SEQ ID NO. 2. In a preferred aspect of (1) to (4), the probe has one of the amino acid sequences of this group or is composed of 2 or more of the amino acid sequences of this group in one fusion protein, wherein the repetition of one or more of the sequences within the fusion protein is also possible. The invention also relates to the nucleic acids which comprise nucleic acid fragments coding for such proteins, preferably DNA sequences and cDNA.

In a preferred aspect of embodiment (2), the tenascin-R probe is human tenascin-R or a homologue/fragment thereof and can be obtained either by preparation from cells of humans or by recombinant production. In particular, as a native TN-R, it is obtainable from cells of neural origin, more preferably from SH-SY5Y neuroblastoma cells. The recombinant preparation of the TN-R fragments, in particular, is preferably effected in correspondingly transformed human Flp-In 293 cells.

A preferred aspect of embodiments (1) and (2) is the performance of the process as a one-step process and/or by selective substrate adhesion to the tenascin-R probe. Thus, single cell suspensions obtained by the enzymatic treatment of the desired central-nervous tissue are sown onto plastic surfaces coated with TN-R proteins or protein fragments. After incubation, preferably for 8-20 hours and preferably in a serum-free medium (what prevents the proliferation of astrocytes and microglial cells), pure oligodendrocyte populations are found on the immobilized TN-R substrates (FIG. 7). Other cell types are present in the cell culture supernatant and can be removed completely by changing the medium. Since only one selection step occurs and the selection phase is short as compared to the duration of the previously usual cultivation for several weeks of mixed glial cultures and other selection methods, the resulting cell yield is high. The reason for this is, inter alia, the fact that all oligodendrocytes (of different differentiation stages) from a primary tissue can be selected on TN-R substrates. In contrast, the isolation of oligodendrocytes from mixed glial cultures is accompanied by a high loss of cells (e.g., when the oligodendrocytes and microglia adhering to an astrocyte monolayer are shaken off and microglial cells are further selected). A comparative example may illustrate this: to obtain a yield of 2-4×10⁶ oligodendrocytes from PO-P2 rodent brains, selection on TN-R substrates from the tissue of one brain in one step suffices, while ten brains and at least 2 weeks of culturing time are required when mixed glial cultures are cultivated.

The isolation process according to the invention according to (1) and (2) allows for the isolation of complete oligodendrocyte populations and is thus also advantageous over immunological selection methods, such as FACS, biomagnetic cell sorting or antibody panning. These only allow for the enrichment of distinct oligodendrocyte populations; oligodendroglial cells not recognized by the antibodies are lost.

Thus, the process according to the invention of (1) and (2) allows for the isolation of oligodendrocyte populations that are often sufficient for further experiments from a single vertebrate, especially from a single rodent. This is of advantage, in particular, if particular effects on mice are examined that occur, for example, in transgenic mice, knockout mice or mice treated with test substances.

The primary tissues used as the starting material of embodiments (1) and (2) can originate from different CNS regions and from different developmental stages. Preferred CNS regions are the brain and individual brain regions (especially forebrain, hindbrain, hippocampus, brain stem), the optical nerve and the spinal chord. The suitable developmental stages include embryonic, fetal, early/late postnatal and adult tissues, preferably early postnatal and adult tissues.

If a single cell suspension is used in the process according to (1) to (3), it contains cells of one or more differentiation stages, preferably a single differentiation stage.

The neural primary tissues used for performing the process according to (1) and (2) originate from lower and higher vertebrates including fish, amphibians, reptiles, birds and mammals, more preferably shark, carp, chicken, rodents including mouse and rat, cattle, pig and human, even more preferably rodents and humans.

The TN-R probe for use in (1) and (2) preferably originates from TN-R of higher and lower vertebrates. Preferably, it is the native TN-R or a fragment of native TN-R.

The recovery of single cell suspensions from primary tissue for use in processes according to embodiment (1) or (2) is effected by methods usual in the art. Thus, the tissue can be converted to tissue fragments and/or single cells in one or more steps mechanically and/or enzymatically. Suitable methods are described in “Zellund Gewebekultur” (T. Lindl, Spektrum Akademischer Verlag, 2002) and

“Current Protocols in Neuroscience” (John Wiley & Sons, Inc., 2004, Ed. 1 Crawley et al.). The thus obtained cells are resuspended in serum-free medium and then contacted with the TN-R probe. After an incubation time which is sufficient for the complete adsorption of the selected cells (1-48 hours, preferably 8-20 hours) under suitable conditions, the non-adherent cells are removed. For further use, the adhered cells may either remain adherent or be detached from the adsorbent enzymatically, preferably by treatment with trypsin or trypsin-EDTA, collagenase, dispase, pronase, Accutase® or other suitable proteinases, especially with Accutase®. Their further use comprises culturing, also on other substrates or plastic surfaces or in other defined media, for obtaining, for example, immature precursor oligodendrocytes or myelin-competent oligodendrocytes.

The process according to (1) and (2) can be employed independently of whether the TN-R probe and the neural primary tissue originate from organisms of the same species. Thus, isolation of oligodendrocytes over species boundaries is possible. TN-R from different vertebrates can be used for the selection of oligoden-drocytes from single cell cultures of other species (Example 3; FIG. 7). Inter alfa, this includes the selection of oligodendrocytes from human, pig, cattle, chicken, mouse, rat, frog and other higher vertebrates for TN-R from a different species (including fish, chicken, mouse, rat, cattle, pig, human etc.).

Further, the process according to (1) and (2) can be employed irrespective of the differentiation stage the selected cells are in (e.g., precursors—immature—mature oligodendrocytes). All cells of a cell type are selected irrespective of its developmental stage.

In one aspect, the selection of defined cell populations from single cell suspensions according to (1) and (2) is effected by isolating the cells bound to the TN-R probe, which are designated for further use. In another aspect, in contrast, it is the supernatant that is freed from these cells by the specific adsorption of cells to the TN-R probe and designated for further use as a defined cell population. In yet another aspect, a modified TN-R or a TN-R fragment which is selective for cells other than the starting protein (native TN-R) is used as the TN-R probe. The TN-R probe in the latter aspect preferably comprises the FN III domains 1 to 8 or 4 to 6, more preferably the proteins encoded by by 1051-3483 in SEQ ID No. 1 (H-TNR-52; human FN III domain 1-8) and by 1573-2945 in SEQ ID No. 1 (H-TNR-S5; human FN III domain 4-6).

In one aspect of embodiment (3), the tenascin-R probe is coupled to a support material by suitable methods for immobilization. Such immobilization may be effected covalently or noncovalently. Such suitable immobilization methods include adequate coupling techniques which leave the specificity of the tenascin-R probe unchanged, such as the covalent cross-linking of the protein with the support material or the immobilization by interaction with a suitable antibody. Preferably, the coupling is effected noncovalently (Example 3), through an antibody or by covalent cross-linking.

In a preferred embodiment of this aspect of embodiment (3), for the isolation of cells from primary tissue of neural origin, support materials, such as cell culture plates, are coated with TN-R or with recombinantly prepared TN-R fragments by incubating the support material, preferably a plastic surface, with a solution of the TN-R probe and subsequently washed. The isolation of ultrapure cell populations from cell suspensions is then effected by selective substrate adhesion. Using TN-R, 100% pure oligodendrocyte preparations could be recovered from early postnatal rodent brains (Example 3): 2×10⁶ oligodendrocytes from a P0-P2 rodent forebrain, 4-6×10⁶ oligodendrocytes from a P5 rodent forebrain, 2×10⁶ oligodendrocytes from a P7-P8 rodent hindbrain. Similar results are possible with recombinantly prepared tenascin-R fragments.

In another embodiment of this aspect, the TN-R probe is immobilized on a plastic surface. This also enables the selective adhesion and isolation of defined cell populations, especially oligodendrocytes, but not of other neural cells (such as astrocytes, microglia or neurons), from the mixed cell populations used as a starting material.

The immobilization is preferably effected by direct contact of the TN-R probe with the support surface. After a sufficient incubation time (1-4 hours), the unbound protein is washed off. The unoccupied binding sites on the surface are subsequently blocked, for example, by incubation with a BSA-containing blocking buffer. The thus coated surfaces can be kept humid until use or be used after drying, the use of undried surfaces being preferred.

In embodiment (3), native TN-R or a fragment of native TN-R are used as a preferred TN-R probe. Also, the use of a mixture of more than one TN-R probe for the coating of the support material is possible.

The process according to embodiment (1) to (3) is suitable for the recovery of neural cells, especially of oligodendrocytes, for the growth of differentiated cells, especially neural cells, in neurobiological and cell-physiological examinations, in biological and clinical research and for diagnostic and therapeutic methods in vitro and in vivo, especially for the preparation of a medicament for cell therapy and for the therapy of neurodegenerative diseases. Further, it can be used for the detection of neurodegenerative diseases.

Embodiment (9) relates to antibodies, preferably monoclonal antibodies according to embodiment (10), which bind to TN-R in at least two species, preferably two vertebrates, preferably the monoclonal antibodies R4 and R6 (Example 5). The latter, in contrast to the antibodies R1 and R2 as described in Pesheva, P. et al. (J. Cell. Biol. 109: 1765-1778 (1989)), show cross-reactivity with different species/vertebrates. In particular, R6 can be used for the detection of TN-R in all classes of vertebrates (fish, amphibians, reptiles, birds and mammals; FIGS. 5A and 5B); R4 recognizes the protein only in higher classes of vertebrates (FIG. 5A and Table 2). R4 and R6 recognize protein epitopes on the TN-R molecule, i.e., they are still active even after glycosidase digestion, which results in cleavage of the sugar residues of the protein.

The cross-activity with different species is caused by the preparation process, namely the fact that a suitable host organism is immunized with tenascin-R from at least two different species, preferably from two different vertebrates, by usual methods and isolated in subsequent screening and purification steps. Suitable host organisms for the immunization include, in particular, non-human mammals, such as rodents (mice, rats etc.), rabbits, guinea pigs, goats etc.

The antibodies according to embodiment (9) are applicable in various immuno-chemical methods based on the detection and/or binding of TN-R (Table 2, fields of application), especially in ELISAs, Western blots, histological and cytological examinations and immunoprecipitations. They are also important in in-vitro assays, since their presence can neutralize the inhibitory effect of the TN-R protein on the neuronal cell adhesion and the axon growth (FIG. 5C). The antibodies react specifically with TN-R in brain extracts or purified TN-R and show no cross-reactivity with other TN proteins. The latter can be concluded from the fact that no reaction takes place with heart or kidney (in mouse containing TN-W, Scherbich, A. et al., J. Cell. Sci. 117: 571-581 (2004)), neonatal skin or skin fibroblast conditioned medium (containing TN-X, Zweers, M. C. et al., Cell Tissue Res. 319: 279-287 (2005)) and TN-C preparations from mouse brain. The reactivity of R4 and R6 is shown in an exemplary manner in FIG. 5.

However, the cross-reactivity of the antibodies according to embodiment (9) exists inasmuch as R1, R2, R4, R5 and R6 react with TN-R from all tested higher vertebrates, and R1, R5 and R6 even react with all species tested (Table 2, FIG. 5B). Therefore, the latter are preferred in one aspect of embodiment (9).

R1, R2, R4, R5 and R6 recognize different protein epitopes on TN-R, which was seen by the enzymatic removal of the N- and O-linked glycoconjugates and determination of the topographic closeness of molecular epitopes by competitive ELISA. This also explains why the antibodies have a different influence on the adhesion and the neurite growth of mouse neurons on TN-R-containing substrate (FIGS. 5C and D, Example 6). These in-vitro tests indicate that epitopes recognized by R4, R5 and R6 (and in part R2) are involved in such processes.

Therefore, preferred antibodies of embodiment (9) are antibodies which are directed against such epitopes.

The antibodies according to embodiment (10) can be prepared by culturing the cell line according to embodiment (11). In particular, the cell lines of embodiment (11) are so-called hybridoma cell lines. These are obtainable, for example, by the immunization of a suitable host organism with TN-R from at least two different species as described above, isolation of splenocytes from the host organism, followed by fusing with suitable primary cells, for example, myeloma cells. Depending on the host organism and on the origin of the primary cells, they are homo- or heterohybridoma cells, the former being preferred.

Further preferred are the monoclonal antibodies according to embodiment (10) of the invention, among which R4, R5 and R6, especially R4 and R6 as produced by the hybridoma cell lines DSM ACC2754 (tn-R4) and DSM ACC2753 (tn-R6) are particularly preferred.

The antibodies of embodiments (9) and (10) of the invention are suitable not only for the immunochemical detection of TN-R, but also for the inhibition of the effect of TN-R in vivo and in vitro.

Due to their interaction with TN-R with respect to neuronal cell adhesion and axon growth, the antibodies according to embodiments (9) and (10) and further according to embodiment (14) can be employed for selectively influencing neural development in vivo and in vitro, for the therapy and prophylaxis of traumatic nerve lesions, and for the preparation of medicaments for selectively influencing neural development and for the therapy and prophylaxis of traumatic nerve lesions. Traumatic nerve lesions are produced, for example, after mechanical damage to nerves. The regeneration of the nerve fibers after such lesions is adversely affected by TN-R (Probstmeier, R. et al., 1 Neurosci. Res. 60: 21-36 (2000); Zhang, Y. et al., Mol. Cell, Neurosci. 17: 444-459 (2001); Becker, C.C. et al., Mol. Cell. Neurosci. 26: 376-389 (2004); Xu, G. et al., J. Neurochem. 91: 1018-1023 (2004)).

Therefore, in embodiment (15), the invention also relates to a process for the therapy and prophylaxis of traumatic neural lesions and for the selective influencing of neural development, comprising the step of administering a suitable amount of antibody according to embodiment (9) or (10) to a patient in need of such treatment. The amount of antibody administered and the necessary dosage will be determined by the attending physician on a case by case basis. It depends, inter alia, on the age, body weight and constitution of the patient on the one hand and on the kind and severity of the disease to be treated on the other hand.

The kit according to embodiment (12) preferably contains the protein defined in embodiment (4) or a stock culture of the cell line for the production of such protein. More preferably, such a kit contains human tenascin-R or its fragments according to the invention and/or a stock culture of cells which are suitable for the recombinant production of these proteins.

A preferred aspect of embodiment (12) is a kit in which the tenascin-R probe has been bound to a support material by an adequate coupling technique as described in the aspects of embodiment (3), and/or which further contains TN-R antibodies (for example, for confirming the immobilization efficiency of the TN-R probe), one or more enzymatic solutions for cell dissociation, optionally agents for the detection of the binding of cells to the tenascin-R probe, buffers and/or culture media. The buffers and media include, in particular, blocking buffers and defined serum-free culture media. Antibodies contained in the kit are preferably antibodies of embodiment (9) or (10).

In embodiment (13), the use of a TN-R fragment or a TN-R fusion protein as defined in embodiment (4) is preferred.

A preferred use of the TN-R probe according to the invention is the use of the TN-R probe for the recovery of oligodendrocytes according to embodiment (13). A related aspect of embodiment (13) is the culturing of differentiated cells from the thus obtained cell populations. The differentiation-promoting effect of the native TN-R has been described (Pesheva, P. et al., 3. Neurosci. 17: 4642-51 (1997)). The fragments of TN-R according to the invention can also have a differentiation-promoting effect on oligodendrocytes of different stages of maturation, especially H-TNR-S3 and H-TNR-S6 (cf. Example 2).

The diagnostic methods according to embodiment (13) can be performed in vivo and in vitro, but preferably in vitro. For the use according to embodiment (13), a tenascin-R probe according to embodiment (4) is preferably used, especially human tenascin-R or its fragments according to the invention. Said tenascin-R probe may have been purified from sources in which it naturally occurs or may have been prepared recombinantly. Preferably, a recombinant tenascin-R probe is used.

Neurodegenerative diseases associated with a loss of oligodendrocytes (by cell death) or myelin, such as multiple sclerosis (MS), are characterized in that a remyelinization cannot take place in the affected regions, or only so to a small extent. This is mainly due to the fact that the existing “traumatic” (i.e., altered due to the action of pathological stimuli) precursor and immature oligodendrocytes are not capable of differentiating/remyelinizing. The invention offers approaches for novel diagnostic and/or therapeutic methods:

For the diagnosis of MS, the quick (within 1-2 days) selection of “traumatic” cells for TN-R probes is particularly suitable, preferably from an animal model or from biopsy samples from patients. This enables the performance of direct examinations of their molecular profile and/or the development of diagnostic markers. For the development of a medicament for cell therapy, “traumatic” oligodendrocytes can be treated with different candidate drugs in order to determine the influence of the latter on the “recovery of traumatic cells”, or the remyelinization potency of such cells.

The method according to the invention is also suitable for the selection of “normal” adult oligodendrocytes that are selected under traumatic conditions in vitro (by adding relevant cytokines or cerebrospinal fluid samples from patients/sick animals) and subsequently treated with candidate drugs before culturing. Such an in-vitro system allows examinations relating to the traumatic alterations occurring in oligodendrocytes that allow conclusions to be drawn to such alterations in vivo.

Further, such cultured oligodendrocytes can serve for the development of diagnostic markers, wherein different stages of traumatic alterations can also be established. The thus obtained oligodendrocytes are also suitable for the screening for candidate drugs that compensate for cell death and/or a lacking myelinization competence of oligodendrocytes under traumatic conditions, or may be employed as a medicament for the cell therapy in vivo.

Finally, the use of a recombinant TN-R fragment, especially the C terminus or a C-terminal fragment, above all the fragments H-TNR-S3 and/or H-TNR-S6 (cf. Example 2), as a medicament or for the preparation of a medicament for the direct cell therapy in neurodegenerative diseases, especially multiple sclerosis, is also possible.

Thus, a preferred aspect of embodiment (13) is the use of the tenascin-R probe for the diagnosis of multiple sclerosis (MS) and for the preparation of a medicament for MS.

Thus, another preferred aspect of embodiment (13) is the use of the tenascin-R probe for the preparation of a medicament for cell therapy and for the therapy of neurodegenerative diseases accompanied by a loss of oligodendrocytes or myelin, especially multiple sclerosis and periventricular leukomalacia (PVL).

Embodiment (16) relates to a process for cell therapy and for the therapy of neurodegenerative diseases accompanied by a loss of oligodendrocytes or myelin, especially multiple sclerosis and periventricular leukomalacia (PVL), comprising the step of administering a pharmacologically sufficient amount of the TN-R probe to a patient in need of such treatment. The amount administered and the necessary dosage will be determined by the attending physician on a case by case basis. It depends, inter alia, on the age, body weight and constitution of the patient on the one hand and on the kind and severity of the disease to be treated on the other hand.

The process (17) for preparing oligodendrocytes from isolated stem cells is preferably performed with neural or non-neural stem cells that have a potential for sulfatide expression. Particularly preferred isolated stem cells are progenitor cells of neural or hematopoietic origin. Thus, human neural stem cells can be selectively differentiated into mature oligodendrocytes in the presence of TN-R (Example 7).

Even more preferably, the process (17) serves for the differentiation of immature oligodendrocytes in vitro. As shown in FIG. 8, immature oligodendrocytes differentiate morphologically under the influence of exogenous substrate-bound TN-R of all tested vertebrates. At the same time, the myelin gene expression is upregulated as can be proven by the quick induction of MBP expression. These effects are particularly strong when TN-R from higher vertebrates is employed. The cell response of the oligodendrocytes is presumably mediated by TN-R interaction with sulfatides and probably includes an autocrine TN-R regulation (Pesheva, P. et al., J. Neurosci. 17: 4642-4651 (1997)). The latter is supported by the fact that the TN-R secretion by oligodendrocytes that were cultured on a TN-R-containing substrate was increased strongly by TN-R from higher vertebrates and slightly less by fish TN-R (FIG. 8B).

For the differentiation of immature oligodendrocytes in vitro on TN-R-coated surfaces, a TN-R concentration of at least 10 μg/ml is preferably employed. Particularly preferred is a TN-R concentration of at least 20 μg/ml, more preferably a TN-R concentration of 20 μg/ml.

Preferably, the process (17) is performed with TN-R from higher vertebrates, more preferably with a TN-R probe according to the invention, even more preferably with native TN-R or a TN-R fragment or fusion protein of embodiment (4).

The hybridoma cell lines to-R4 (producer of antibody R4) and to-R6 (producer of antibody R6) were deposited on Dec. 2, 2005, under the accession Nos, DSM ACC2754 and DSM ACC2753, respectively, with the DSMZ, Deutsche Sammlung von Mikroorganismen and Zellkulturen GmbH, Mascheroder Weg 1, 38124 Braunschweig, Germany.

The invention is further illustrated by means of the following Examples. However, they do not limit the scope of protection of the invention.

### EXAMPLES

### Solutions/Media Employed:

1. HBSS (Hank's Balanced Salt Solution) (Sigma)
2. Enzymatic solutions for cell dissociation:

1% (w/v) trypsin solution: 1% (w/v) trypsin (cell culture tested), 0.1% (w/v) DNase I, 1 mM EDTA, 0.8 mM MgSO₄, 10 mM HEPES in HBSS (Ca/Mg-free). DNase solution: 0.05% (w/v) DNase I, 10 mM HEPES in SME (Basal Medium Eagle).
3. Defined serum-free medium:

DMEM (Sigma) supplemented with insulin

(10 μg/ml), progesterone (0.06 μg/ml), triiodothyronine (0.34 μg/ml), L-thyroxine (0.52 μM), putrescine (16 μg/ml), sodium selenite (0.22 μM), transferrin (0.1 mg/ml), HEPES (25 mM), gentamicin (25 μg/ml), penicillin (100 units/mil) and streptomycin (0.1 mg/ml).
4. Blocking buffer:

2% (w/v) BSA (bovine serum albumin, fatty-acid-free) in PBS (150 mM NaCl, 8 mM Na₂HPO₄, 17.4 mM NaH₂PO₄), pH 7.2, heat-inactivated (for 20 minutes at 70° C.).

Tissue extracts and Western blots

Tissue samples were homogenized in PBS or TES (see Example 1) with or without 1% Triton® X-100 for 2 hours at 4 ° C. All buffers contained spermidine and protease inhibitors (cf. Example 1). Insoluble material was separated off by sedimentation. Tissue extracts from fish (50 μg of protein/lane) and other vertebrates (20 μg of protein/lane) were separated by SDS-PAGE under reducing conditions over 7% polyacrylamide gels and either subjected to silver staining or analyzed by a Western blot with TN-R-specific antibodies. In the Western blots, alkaline phosphatase or horseradish peroxidase (HRP) conjugated secondary antibodies (Promega; Roche Diagnostics) served for detection (Pesheva, P. et al., J. Neurosci. Res. 51: 49-57 (1998)).

Enzymatic treatment of TN-R: For the enzymatic removal of N-linked oligosaccharides, purified TN-R proteins were treated with N-glycosidase F or H (Roche Diagnostics) as described (Pesheva, P. et al., J. Cell. Biol. 109: 1765-1778 (1989)). For the enzymatic removal of O-linked GAGs, the TN-R proteins were treated with chondroitinase ABC or heparinase (Sigma) as described (Probstmeier, R. et al., Brain Res. 863: 42-51 (2000)).

### Cell Cultures

Primary cultures of hindbrain neurons (in serum-free Fischer medium; Pesheva, P. et al., Neuron 10: 69-82 (1993)), oligodendrocytes (in serum-free Sato medium; Pesheva, P. et al., J. Neurosci. 17: 4642-4651 (1997)) and skin fibroblasts (in DMEM 10% FCS) were prepared as described. For the examination of neurite growth, hindbrain neurons (1×10⁶ cells/ml) were cultured on the test substrates in a serum-free Fischer medium.

### Example 1

Purification of TN-R proteins by Immunoaffinity Chromatography

The purification of TN-R proteins from adult brain (shark, carp, chicken, mouse, rat, cattle, pig, human) was effected by immunoaffinity chromatographic methods (FIG. 6). Thus, the starting tissue was macerated with a urea-containing buffer (20 mM Tris-HCl, 10 mM EDTA, 10 mM EGTA, 1 M urea, pH 7.9; including 1 mM spermidine and the following protease inhibitors: 1 μM aprotinin, 5 μM SBTI (trypsin inhibitor from soybean), 1 mM PMSF (phenylmethyisulfonylfluoride), iodoacetamide (19 μg/ml), type III trypsin inhibitor from egg white (10 μg/ml)) at 4° C. for 2 hours, followed by pelletizing insoluble fractions at 30,000 g for 30 minutes. The supernatant was precipitated with 40% (w/v) ammonium sulfate, and precipitated fractions were collected by a centrifugation step at 30,000 g. The precipitate was subsequently dissolved in 20 mM Tris-HCl, 1 mM EDTA, 1 mM EGTA, 150 mM NaCl, pH 7.2, and dialyzed against the same buffer. Undissolved fractions were removed by centrifugation for one hour at 100,000 g and 4 ° C.

Alternatively, the following maceration method was used: Homogenization of the tissue in TES buffer (10 mM Tris-HCl, 150 mM NaCl, 10 mM EDTA, pH 7.4; including 1 mM spermidine and the above stated protease inhibitors) and incubation over night at 4° C. Thereafter, undissolved fractions were removed by centrifugation for one hour at 100,000 g and 4° C. The supernatant of the respectively latest centrifugation step was subsequently used for the further purification of TN-R proteins by immunoaffinity chromatography. Thus, the supernatants were passed through column matrices to which monoclonal TN-R antibodies were bound. These were monoclonal antibodies designated as R1, R2 (Pesheva, P. et al., J. Cell. Biol. 109: 1765-1778 (1989)), R4 or R6 (see Example 5). These antibodies were coupled to CNBr-activated Sepharose ®4B. After the passage of the centrifugation supernatants, the antibody columns were washed first with 20 mM Tris-HCl, 1 mM EDTA, 1 mM EGTA, 0.5 M NaCl, 0.5% (v/v) Triton® X-100, pH 7.2, then with PBS (phosphate-buffered saline, pH 7.2). The TN-R proteins bound to the antibodies were detached from the column with a basic elution buffer (0.1 M diethylamine, 0.1 M NaCl, 1 mM EDTA, 1 mM EGTA, pH 11.2). The eluate was neutralized immediately and dialyzed against PBS.

### Example 2

### Preparation of Human TN-R Fragments

Much like the other known TN-R proteins, human TN-R protein is composed of different distinct domains (Carnemolla, B. et al., 3. Biol. Chem. 271: 8157-60 (1996)). Starting with a cystein-rich region at the N terminus, followed by 4,5-EGF-like domains and 8 FN-III-like domains (wherein another domain may be present between the 5th and 6th domains when there is a corresponding alternative splicing, which then results in 9 FN-III-like domains), the molecule ends at the C terminus with a fibrinogen-like domain. With 9 EN-III-like domains, the published human TN-R sequence comprises 4716 bases (SEQ ID No. 1; NCBI nucleotide NM_{—}003285). Of these, the coding region corresponds to the segment between bases 82 to 4158, and the signal peptide (for the secretion of the TN-R protein) corresponds to the base region 82 to 150. For the preparation of recombinant eukaryotically expressed TN-R protein fragment, the following DNA sequences were selected in accordance with individual domain regions:

### “H-TNR-S1”: by 151-1065

(region: “Cys region” to “EGF-like domains” (incl.))

### “H-TNR-S2”: by 1051-3483

(region: “FN-III-like domain 1” to “EN-III-like domain 8” (incl.))

### H-TNR-S3″: by 3439-4155

(region: “fibrinogen-like domain” (incl.))

### “H-TNR-S4”: by 151-1599

(region: “Cys” region to “FN-III-like domain 3 (incl.))

### “H-TNR-S5”: by 1573-2945

(region: “FN-III-like domain 4” to “FN-III-like domain 6” (incl.))

### “H-TNR-S6”: by 2926-4155

(region: “FN-III-like domain 7” to “fibrinogen-like domain” (incl.))

In total, the fragments H-TNR-S1 to H-TNR-S6 cover the whole TN-R protein.

For the recovery of human TN-R-specific mRNA, the human neuroblastoma cell line SH-SY5Y was used (Woodworth, A. et al., J. Biol. Chem. 279: 10413-21 (2004)). Total RNA was purified from these cells using triazole reagent (Invitrogen) according to the manufacturer's instructions. The cDNA synthesis from these RNA preparations was effected using “random hexamer” primers or oligo(dT) primers by means of the SuperScript II system (Invitrogen) according to the manufacturer's instructions. For the preparation of H-TNR-S1-to H-TNR-S6-specific cDNA fragments, the following primers were used:

H-TNR-S1
SEQ ID No. 3: upstream:
TCC ATG ATC AAG CCT TCA GAG TG (bp 151-173)
SEQ ID No. 4: downstream:
AGG GGC AAC TGC TGA GCA GT (bp 1046-1065)
(product length: 915 bp)
H-TNR-S2
SEQ ID No. 5: upstream:
TCA GCA GTT GCC CCT CCA GAG G (bp 1051-1072)
SEQ ID No. 6: downstream:
ATG AGG GAA CAC CCG GCC TCC (bp 3463-3483)
(product length: 2433 bp)
H-TNR-S3
SEQ ID No. 7: upstream:
ATC ACC TCC ACC GCT TTC ACC (bp 3439-3459)
SEQ ID No. 8: downstream:
GAA CTG TAA GGA CTG CCG TTT TCT (bp 4132-4155)
(product length: 717 bp)
H-TNR-S4
SEQ ID No. 9: upstream:
TCC ATG ATC AAG CCT TCA GAG TG (bp 151-173)
SEQ ID No. 10: downstream:
GCC GTC AAT GAC TGT GGA GAC (bp 1579-1599)
(product length: 1449 bp)
H-TNR-S5
SEQ ID No. 11: upstream:
GCC AGC GTC TCC ACA GTC ATT G (bp 1573-1594)
SEQ ID No. 12: downstream:
GTT GTC CAT GGC TGT GTG CAC A (bp 2925-2946)
(product length: 1374 bp)
H-TNR-S6
SEQ ID No. 13: upstream:
GTG CAC ACA GCC ATG GAC AA (bp 2926-2945)
SEQ ID No. 14: downstream:
GAA CTG TAA GGA CTG CCG TTT TC (bp 4133-4155)
(product length: 1230 bp)

The PCR fragments obtained were cloned into the pcsecTag/FRT/V5-His-TOPO vector (Invitrogen) by means of the TOPO TA cloning system (Invitrogen), which makes use of the overhanging A residues of the PCR products when Taq polymerase is used, according to the manufacturer's instructions. After stable integration into eukaryotic Flp-In cell lines (see below), this vector allowed for the secretion of the desired protein fragment provided with a 6× His peptide at the C terminus into the cell culture supernatant. PolyHis-bearing protein fragments were purified by nickel chelate chromatography from collected cell culture supernatants.

Flp-In 293 cells (Invitrogen), which are derived from the human kidney cell line HEK 293, were used as producers of the protein fragments. In Flp-In cells, the plasmid pFRT/IacZeo (Invitrogen) is stably integrated. This vector contains an FRT region which is specifically recognized by Flp recombinase. When the Flp 293 cells are simultaneously transfected with the pOG44 plasmid, which enables the expression of Flp recombinase, and the pcsecTag/FRT/V5-His-TOPO vector, which bears the base sequence of the desired protein fragment, an incorporation of those fractions of the pcsecTag/FRT/V5-His-TOPO vector that are necessary for the preparation of the secreted protein fragment occurs at the FRT region.

### Example 3

Selective Purification of Oligodendrocytes from CNS Tissue of Mammals Using Native Tenascin-R

As the starting material, postnatal mouse brains (either the total brain or isolated forebrain and hindbrain regions or preparations of the optical nerve) of the age stages postnatal day 0 (P0) to adult were used. After mechanical comminution in accordance with origin and age, isolated brain regions were treated with 0.5 to 1% (w/v) trypsin solution (P0 to P2 brains: with 0.5% (w/v) trypsin solution for 12 min at room temperature (RT), P5 brains: with 1% (w/v) trypsin solution for 15 min at RT, P8 brains: with 1% (w/v) trypsin solution for 20 min at RT, and adult brains: with 1% (w/v) trypsin solution for 30 min at RT). After a substantial volume of HBSS was added, the tissue portions were pelletized at 600 g for 10 minutes at 4° C.

For the recovery of individual cells, the pelletized tissue pieces were taken up in DNase solution and pipetted up and down repeatedly in a Pasteur pipette having a narrowed tip diameter. The coarse cell suspension obtained was diluted in a five-to 10 fold volume of serum-free medium (supplemented with 0.2% (w/v) heat-inactivated bovine serum albumin) and incubated on ice for 5 minutes. The supernatant containing the single cell suspension was centrifuged at 600 g for 10 min at 4° C., and the pelletized single cells were resuspended in serum-free medium. The thus obtained single cell suspensions contained all cell types present in the corresponding brains/brain regions (neurons, astrocytes, oligodendrocytes, microglia, meningial and endothelial cells).

For the recovery of pure oligodendrocyte populations, the single cell suspensions presented in the preceding paragraph (2×10⁶ cells/ml in serum-free medium) were cultured on cell culture plates coated with tenascin-R protein (see below) and cultured in a CO₂ incubator (5% CO₂) for 8-20 hours. Non-adherent cells were washed away with HBSS, and adherent cells were further cultured in serum-free medium. Subsequently, the cells adherent on TN-R substrates were incubated with a GalC-specific monoclonal mouse antibody (O1; Bansai, R. et al., J. Neurosci. Res. 24: 548-557 (1989)) (30 min at RT). After fixing the cells with 4% (v/v) paraformaldehyde in PBS for 10 min at RT, the binding of the O1 antibody onto the cells was detected by incubation with cyanine-3- or FITC-coupled anti-mouse Ig antibodies (20 min at RT) by fluorescence microscopy. On the substrate, there remained 99±1% of GaIC-stainable cells, i.e., only oligodendrocytes (FIG. 7, bottom line).

The cells obtained by this one-step process could be detached from the substrate for further intended uses by treatment with Accutase® (Sigma) and cultured further on other substrates/plastic surfaces or in other defined media for the recovery of, for example, immature precursor oligodendrocytes or myelin-competent oligodendrocytes.

For the preparation of substrates of tenascin-R proteins/protein fragments, plastic surfaces (cell culture plates, flasks etc.) were incubated with the corresponding proteins/protein fragments (20-40 μg/ml in PBS) for 1-2 hours at 37° C., washed with PBS, then incubated with blocking buffer (1 hour at 37° C.) and subsequently washed again with PBS (2-3 times). For the preparation of TN-R substrates for the recovery of pure oligodendrocyte populations, TN-R proteins from shark, carp, chicken, mouse, rat, cattle, pig or human could be used. At least the substrates prepared from rodent TN-R can also be dried after the coating without thereby losing the specific adhesive properties of the TN-R proteins for oligodendrocytes.

### Example 4

Selective Purification of Oligodendrocytes from CNS Tissue of Mammals by Means of Tenascin-R Fragments

For the selection of pure oligodendrocyte populations from CNS tissue by means of recombinantly prepared TN-R fragments, the steps described in Example 3 for the recovery of single-cell suspensions from postnatal brain tissue and for the selection of oligodendrocytes on cell culture plates coated with TN-R fragments from the cell suspension under serum-free culturing conditions were essentially used. The oligodendroglial cells obtained by such one-step process can be detached from the substrate for further intended uses and further proliferated or examined on other substrates or under different culture conditions.

For the preparation of substrates from recombinantly prepared TN-R fragments, plastic surfaces are coated with the corresponding protein fragments originating from the C terminus of human TN-R that contain amino acid sequences of the FNG domain and/or parts thereof (10-20 μg/ml in PBS for 2 hours at 37° C.). Alternatively, the corresponding TN-R fragments are covalently coupled to supports, for example, by an N-alkylcarbamate linkage of amino groups of the protein fragment to 1,1′-carbonyldiimidazole-activated matrices (i.e., plastic surfaces or biopolymers). The substrate supports are subsequently washed with PBS, incubated with blocking agent (for 1 h at 37° C.) and finally washed again with PBS.

### Example 5

### Preparation of Monoclonal TN-R Antibodies

The monoclonal TN-R antibodies R1 and R2 have already been characterized (R1=antibody from clone 597, R2=antibody from clone 596 in Pesheva, P. et al., J. Cell. Biol. 109: 1765-1778 (1989)). They were produced against chicken brain glycoproteins and recognize various vertebrate TN-R, including from chicken and human (Table 2). The monoclonal TN-R antibodies R4, R5 and R6 were prepared by immunization with an equimolar mixture of chicken TN-R and human TN-R as an antigen in BALB/c mice (3 subcutaneous injections at 2 week intervals with 5 μg of protein/mouse). The mentioned TN-R proteins had previously been recovered from adult brain tissue by immunoaffinity chromatographic purification through column matrices to which the R2 antibodies were coupled. Hybridoma clones obtained by the fusion of splenocytes originating from mice immunized with such TN-R proteins with mouse myeloma cells (myeloma cell line P3X63/Ag8) were screened in ELISA assays against chicken, mouse and human TN-R. For this purpose, microtitration plates were coated with mouse TN-R or an equimolar mixture of chicken and human TN-R (0.5 μg/ml in 0.1 M NaHCO₃ over night at 4° C.) and incubated with hybridoma supernatants (2 hours at 37° C.). The binding of the cross-reactive antibodies present in these supernatants was detected by incubation with peroxidase-coupled anti-mouse Ig antibodies. The specificity of positive hybridoma clones for TN-R was independent of the origin of the TN-R protein, as could be shown by further ELISA and Western blot analyses of TN-R proteins and brain extracts purified by immunoaffinity.

The purification of the antibodies was effected by separating the supernatants of the hybridoma cultures over protein G/sepharose columns (Amersham).

Like R1 and R2, the antibodies R4, R5 and R6 belonged to the IgG1 subclass of immunoglobulins and recognized TN-R proteins in different classes of vertebrates (FIG. 5 and Table 2): fish (R5, R6), amphibians (RS, R6), reptiles (R4, R5, R6), birds (R4, R5, R6) and mammals (R4, R5, R6). Thus, R4 recognized only the TN-R in higher vertebrate classes. The results for R1, R2, R4, R5 and R6 are summarized in Table 2. In higher vertebrates, R1 recognized only the 180 kD form of the TN-R protein.

None of the antibodies reacted with other ECM proteins in addition to TN-R (such as TN-C, fibronectin, laminin, vitronectin or collagens; cf. FIG. 5E).

Even after the cleavage of the sugar residues from the TN-R proteins by glycosidase digestion, R4 and R6 were still active, i.e., they recognized protein epitopes on the TN-R molecule.

Table 2 summarizes the results of several ELISA and Western blot analyses: While R2 and R4 mainly react with TN-R from higher vertebrates, R1, R5 and R6 recognize all vertebrate species tested. In higher vertebrates, which have both the 160 kD and the 180 kD form of TN-R, R1 mainly recognizes the 180 kD form. The antibodies can be employed in different immunochemical processes (Table 2). Further, R2, R4, R5 and R6 interfere in vitro with the inhibitory effect of TN-R on neuronal cell adhesion and axon growth by neutralizing this effect (FIGS. 5C and 5D).

TABLE 2
Cross-reactivities of the monoclonal TN-R antibodies (R1, R2, R4, R5, R6)
with various vertebrates; fields of application.

| Vertebrate class/family | TN-R proteins | R1 | R2 | R4 | R5 | R6 |
|---|---|---|---|---|---|---|
| Chondrichthyes | | | | | | |
| Squalidae (shark) | 220 kD | x | | | x | x |
| Ostheichthyes | | | | | | |
| Cyprinidae (carp) | 170 kD | x | | | x | x |
| Salmonidae (trout) | 170 kD | x | | | x | x |
| Amphibia | | | | | | |
| Salamandridae (salamander) | 180 kD | x | x | | x | x |
| Ranidae (frog) | 160-180 kD | x | x | | x | x |
| Reptilia | | | | | | |
| Colubridae (grass snake) | 160-180 kD | x | x | x | x | x |
| Testudinidae (tortoise) | 160-180 kD | x | x | | x | x |
| Aves | | | | | | |
| Phasianidae (chicken) | 160-180 kD | x | x | x | x | x |
| Columbidae (pigeon) | 160-180 kD | x | x | x | x | x |
| Mammalia | | | | | | |
| Erinaceidae (hedgehog) | 160-180 kD | x | x | x | x | x |
| Muridae (mouse, rat) | 160-180 kD | x | x | x | x | x |
| Sciuridae (mole) | 160-180 kD | x | x | x | x | x |
| Leporidae (rabbit) | 160-180 kD | x | x | x | x | x |
| Bovidae (cattle) | 160-180 kD | x | x | x | x | x |
| Suidae (pig) | 160-180 kD | x | x | x | x | x |
| Homo sapiens | 160-180 kD | x | x | x | x | x |
| Fields of application | | | | | | |
| ELISA | native | x | x | x | x | x |
| Immunocytochemistry | native/fixed | x | x | x | x | x |
| Immunohistochemistry | native/fixed | x | x | x | x | x |
| Western blot | denatured | ± | x | x | x | x |
| Immunoprecipitation | native | x | x | x | x | x |
| Interference with TN- | | — | x | x | x | x |
| R-mediated inhibition | | | | | | |
| of neuronal adhesion | | | | | | |

### Example 6

### Cell Adhesion and Neurite Growth Tests

For short and long term adhesion tests, either TN-R alone, TN-R in admixture with other ECM proteins or protein fragments, or a TN-R coat on PLL substrate was prepared as described (Pesheva, P. et al., Neuron 10: 69-82 (1993); Pesheva, P. et al., J. Cell. Sci. 107: 2323-2333 (1994)). For neurite growth tests, laminin in admixture with BSA (control protein) or TN-R (ratio 20:20 μg/ml for each protein) was applied as a coat to cell culture plates and incubated at 37° C. for 60 min. For cell adhesion tests, either TN-R alone (20 μg/ml in PBS) or BSA or TN-R admixed with laminin, fibronectin and fibronectin fragments (ratio 20:20 μg/ml for each protein) was applied as a coat to plastic cell culture plates and incubated at 37° C. for 60 min. For the cell adhesion tests, cultured cells (see above, cell cultures) were detached from the cell culture plate by mild treatment with Accutase (PAA Laboratories; 10 min at RT) or 0.01% trypsin (Sigma; 5 min at RT). Then, single-cell cultures (1×10⁶ cells/ml) in the respectively suitable medium were plated onto the test substrates. For quantitative analyses, cells adhering to the different substrates tested were counted in microscopic fields of 800 μm² with the image analysis software AxioVision (Zeiss). Mean values±standard deviation were formed from the results of five different microscopic fields.

### Example 7

Differentiation of Oligodendrocytes from Human Neural Stem Cells

Human neural stem cells (Cambrex, human neural progenitors: PT-2599; 3×10⁶ cells/ml) were proliferated as neurospheroids in NPMM (Neural Progenitor Maintenance Medium, Cambrex, CC-3209) for 7 days (at 37° C. and 5% CO₂) in cell culture flasks (T-75). The medium was changed every 2-3 days. Subsequently, the neurospheroids containing the stem cells were plated onto cell culture plates coated with laminin (20 μg/ml) (1-2 neurospheroids/plate) and proliferated by culturing in a defined serum-free medium (DMEM/Ham's F12, N2 Supplement (Invitrogen), 50 ng/ml bFGF, 10 ng/ml PDGF) for a minimum of 7 days. The medium was half renewed every 2 days. These culture conditions led to the neural stem cells being preprogrammed to a dominantly glial phenotype (detectable by the expression of sulfatides). The cells were subsequently detached from the cell culture plates by treatment with Accutase (PAA, 10 min at RT), taken up in a defined medium (1×10⁶ cells/ml in DMEM, N2 supplement, 10 ng/ml T3 (triiodothyronine, Sigma)) and plated onto cell culture plates coated only with poly-D-lysine (PDL, Sigma) or coated with PDL and TN-R (20 μg/ml). The subsequent incubation at 37° C. and 5% CO₂ had the effect that dominantly mature oligodendrocytes had formed after 5 days in the presence of TN-R, but not in the presence of PDL only, as detected by the expression of MBP (myelin basic protein).

1. A process for the isolation and purification of neural cells from neural primary tissue of vertebrates, comprising selecting the cells from a single cell suspension by means of a probe containing tenascin-R (“tenascin-R probe”), which comprises tenascin-R compounds selected from native tenascin-R (TN-R) as well as homologues and fragments thereof and fusion proteins of such compounds.

2. The process according to claim 1, wherein
(i) said tenascin-R compound of said tenascin-R probe is a recombinant tenascin-R compound; and/or
(ii) said TN-R originates from vertebrates; and/or
(iii) said tenascin-R probe contains further functional peptide or protein sequences and/or is coupled to a support.

3. The process according to claim 1, wherein
(i) said native tenascin-R is human tenascin-R and/or has the amino acid sequence of SEQ ID No. 1 or is a substitution, deletion and/or addition mutant thereof; and/or
(ii) said tenascin-R fragment comprises the C terminus of native tenascin-R or a substitution, deletion and/or addition mutant thereof; and/or
(iii) said tenascin-R fragment comprises the amino acid residues 1287 to 1358 of SEQ ID No. 2 or a substitution, deletion and/or addition mutant thereof; and/or
(iv) said tenascin-R fusion protein comprises a tenascin-R component comprising native tenascin-R, a tenascin-R fragment or a tenascin-R mutant, and a functional component comprising further functional peptides or proteins; or is composed of two or more, functional tenascin-R components as defined above.

4. The process according to claim 3, wherein said tenascin-R fragment is a peptide having the sequence of the amino acid residues 1287-1358, 1120-1358, 1136-1358 or 949-1358 of SEQ ID No. 1.

5. The process according to claim 1, wherein
(i) said process is suitable for the isolation and purification of glial cells; and/or
(ii) said vertebrate primary tissue originates from lower or higher vertebrates; and/or
(iii) the isolation of the cells is effected by selective substrate adhesion to the tenascin-R probe and/or in a single purification step.

6. The process according to claim 1, wherein said single-cell suspension
(i) is prepared from embryonic, fetal, early or late postnatal and/or adult tissue; and/or
(ii) is prepared from tissue from different regions of the nerve system; and/or
(iii) contains cells of one or more differentiation stages.

7. The process according to claim 1, wherein
(i) said tenascin-R probe is bound to a support material by non-covalent interactions or by another adequate coupling technique which does not change the specificity of the tenascin-R probe; and/or
(ii) said single cell suspension is contacted with said tenascin-R probe so that tenascin-R-binding cells present in said single cell suspension become bound to said probe; and/or
(iii) isolation of these cells from the cell culture is effected by specific binding of neural stem cells from said single cell suspension to said tenascin-R probe, the unbound cells are removed, and optionally the cells bound to the support material through said tenascin-R probe are subsequently detached from the support material by trypsinization, incubation with Accutase® or another adequate method; and/or
(iv) the bound cells are detected by immunological methods; and/or
(v) the process is effected in vitro.

8. The process according to claims 1, which is suitable
(i) for obtaining neural cells, for growing differentiated cells, in neurobiological and cell-physiological examinations, in biological and clinical research and for diagnostic and therapeutic processes in vitro and in vivo; and/or
(ii) for the detection of neurodegenerative diseases.

9. A tenascin-R fragment or tenascin-R fusion protein, wherein said tenascin-R fragment comprises the C terminus of native tenascin-R or a substitution, deletion and/or addition mutant thereof and/or comprises the amino acid residues 1287 to 1358 of SEQ ID No. 2; and said tenascin-R fusion protein comprises one or more tenascin-R components selected from native tenascin-R, tenascin-R fragments and tenasin-R mutants and one or more functional components comprising one or more functional peptides or proteins.

10. The tenascin-R fragment or tenascin-R fusion protein according to claim 9, which has an amino acid sequence selected from the amino acids 1287-1358, 1120-1358, 1136-1358 or 949-1358 in SEQ ID No. 2.

11. A DNA which codes for a tenascin-R fragment or tenascin-R fusion protein according to claim 9.

12. A vector which comprises a DNA according to claim 11.

13. A host organism expressing a DNA according to claim 11.

14. A process for preparing a tenascin-R fragment or tenascin-R fusion protein comprising the step of culturing said host organism according to claim 13.

15. An antibody obtainable by the immunization of a suitable host organism with tenascin-R from at least two different species and/or which binds to tenascin-R from at least two different species.

16. The antibody according to claim 15, which is monoclonal.

17. A cell line or hybridoma cell line which produces a monoclonal antibody according to claim 16.

18. Method of using the antibody according to claim 15
(i) for the immunochemical detection of TN-R;
(ii) for inhibiting the effect of TN-R;
(iii) for influencing the neural development; and
(iv) for preparing medicaments for the therapy and prophylaxis of traumatic nerve lesions and medicaments for selectively influencing the neural development.

19. A method for the therapy and prophylaxis of traumatic nerve lesions of for selectively influencing the neural development, comprising the step of administering a pharmacologically sufficient amount of the antibody according to claim 15 to a human or animal patient in need of such treatment.

20. A kit for the isolation and purification of neural cells comprising
(i) a tenascin-R probe as defined in claim 29; and/or
(ii) a vector which codes for the tenascin-R probe as defined in (i); and/or
(iii) a stock culture of a cell line which is suitable for expressing said tenascin-R probe.

21. The kit according to claim 20, wherein
(i) said probe is bound to a support material; and/or
(ii) the kit further comprises tenascin-R antibodies; and/or
(iii) the kit further comprises enzymatic solutions for cell dissociation, buffers and/or culture media.

22. Method of using a tenascin-R probe as defined in claim 29 for obtaining neural cells, for growing differentiated cells, in neurobiological and cell-physiological examinations, in biological and clinical research and for diagnostic and therapeutic processes in vitro and in vivo.

23. A process for cell therapy or for the therapy of neurodegenerative diseases accompanied by a loss of oligodendrocytes or myelin, comprising the step of administering a tenascin-R probe as defined in claim 29 to a human or animal patient.

24. A process for preparing oligodendrocytes from isolated stem cells in vitro by incubating the stem cells in the presence of a tenascin-R probe as defined in claim 29.

25. The process according to claim 24, wherein said isolated stem cells are neural or non-neural stem cells which have the potential for sulfatide expression.

26. The process according to claim 2, wherein said TN-R originates from one of rats, mice and humans.

27. The antibody according to claim 16, which is produced by the hybridoma cell line DSM ACC2754 or DSM ACC2753.

28. The cell line or hybridoma cell line according to claim 17, which is hybridoma cell line DSM ACC2754 or DSM ACC2753.

29. A tenascin-R probe, which comprises tenascin-R compounds selected from native tenascin-R (TN-R) as well as homologues and fragments thereof and fusion proteins of such compounds.
